(19) United States
(12) Patent Application Publication
(10) Pub. No.: US 2025/0336477 A1
(43) Pub. Date: Oct. 30, 2025

(54) METHOD, APPARATUS, DEVICE AND STORAGE MEDIUM FOR DETECTING MRD LESIONS

(71) Applicants: GENECAST (BEIJING) BIOTECHNOLOGY CO., LTD., Beijing (CN); GENECAST PRECISION MEDICAL DIAGNOSTIC LABORATORY WUXI CO., LTD., Jiangsu (CN)

(72) Inventors: Yiqian LIU, Jiangsu (CN); Yaxi ZHANG, Jiangsu (CN); Lingran MA, Jiangsu (CN); Rui FAN, Jiangsu (CN); Jianing YU, Jiangsu (CN); Zhencheng SU, Jiangsu (CN); Weizhi CHEN, Jiangsu (CN); Yu HUANG, Jiangsu (CN); Bo DU, Jiangsu (CN)

(73) Assignees: GENECAST (BEIJING) BIOTECHNOLOGY CO., LTD., Beijing (CN); GENECAST PRECISION MEDICAL DIAGNOSTIC LABORATORY WUXI CO., LTD., Jiangsu (CN)

(21) Appl. No.: 19/230,097

(22) Filed: Jun. 6, 2025

Related U.S. Application Data
(63) Continuation of application No. PCT/CN2023/088612, filed on Apr. 17, 2023.

(30) Foreign Application Priority Data
Dec. 30, 2022 (CN) 202211721580.4

(51) Int. Cl. G16B 35/00; G16B 30/00

(52) U.S. Cl. CPC G16B 35/00; G16B 30/00

(57) ABSTRACT

The current application reveals a method, apparatus, device, and storage medium for detecting micro residual lesions, falling within the domain of medical detection technology. This method is based on differentiated deep whole-exome/targeted drug sequencing and tissue-blood cell-plasma co-capture technology, and 100,000× ultra-high depth personalized/high evidence hotspot combination panel sequencing to evaluate tiny residual lesions and tumor evolution/second primary in plasma samples. It resolves the challenges of existing techniques, such as elevated tissue detection thresholds, restricted tracking locations, inadequate detection sensitivity and precision, or elevated costs when ctDNA concentrations in the bloodstream are minimal. Furthermore, it surmounts the challenge of simultaneously achieving personalized tracking detection and monitoring tumor evolution or second/primary detection. It markedly boosts the precision of forecasting the likelihood of recurrence following patient therapy within a restricted budget.

### CROSS-REFERENCE TO RELATED APPLICATION

This application is a continuation of international application of PCT application serial no. PCT/CN2023/088612, filed on Apr. 17, 2023, which claims the priority benefit of China application no. 202211721580.4, filed on Dec. 30, 2022. The entirety of each of the above-mentioned patent applications is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Jul. 2, 2025, is named 155450US-sequencing_listing and is 46,645 bytes in size.

### TECHNICAL FIELD

The current application pertains to the domain of gene detection technology, and more specifically, it relates to a method, apparatus, device, and storage medium for detecting MRD lesions.

### BACKGROUND

Assessment of MRD (minimal/measurable/molecular residual disease) guided by circulating tumor DNA (ctDNA) can identify patients with MRD more effectively than traditional clinical or imaging methods, and offers greater sensitivity and specificity in predicting the risk of recurrence.

In the related art, for example, a Chinese invention patent with publication number CN112236535A describes a method for cancer detection and monitoring with the aid of personalized detection of circulating tumor DNA, which is used to detect single nucleotide variants in breast cancer, bladder cancer or colorectal cancer, and generates an amplicon set by performing a multiple amplification reaction on nucleic acids, the nucleic acids are separated from a blood or urine sample or a portion thereof from a patient who has been treated for breast cancer, bladder cancer or colorectal cancer, wherein each amplicon in the set spans at least one single nucleotide variant locus in a set of patient-specific single nucleotide variant loci associated with breast cancer, bladder cancer or colorectal cancer; and determines the sequence of at least one segment of each amplicon in the set, wherein the at least one segment contains a patient-specific single nucleotide variant locus, wherein the detection of one or more patient-specific single nucleotide variants indicates early recurrence or metastasis of breast cancer, bladder cancer or colorectal cancer.

However, the detection method above uses nucleic acids in blood or urine as input samples for multiple amplification reactions, which cannot accurately remove repetitive sequences, and high cycle number amplification may introduce amplification errors. In addition, this method uses conventional WES panels to determine tissue sites, and does not focus on monitoring high-evidence-level genes and sites, which are areas with high frequency and clinical evidence in the general tumor patient database. Furthermore, this method only performs personalized panel tracking and is unable to monitor second primary mutations or tumor evolution mutations that may be hidden in blood samples.

### SUMMARY

### 1. Purpose

The current application aims to provide a method, apparatus, device, and storage medium for detecting MRD lesions to solve one of the technical problems mentioned in the above background technology section.

### 2. Technical Solutions

To address the aforementioned issues, the technical solutions implemented in this application are as follows:

As a primary feature of the current application, it offers a technique for identifying MRD lesions, grounded in second-generation sequencing technology. This method encompasses the subsequent steps:

S1, obtain WDC sequencing data of patient tumor tissue DNA and blood cell DNA, that is: construct tumor tissue DNA library and blood cell DNA library respectively; mix the two libraries with equal mass ratio, and use WDC probe for hybridization capture to obtain captured DNA library, wherein WDC probe is a mixed probe formed by mixing whole exome sequencing probe (WES probe) with targeted drug gene panel in a ratio of 1:(2˜8); sequence the captured DNA library to obtain WDC sequencing data of tumor patients. The WDC probe can achieve differentiation in sequencing depth, that is, the effective depth ratio of WES other regions:tumor-related gene regions:targeted drug gene regions can be 1:(1.5-3):(2-6), which can reduce the detection limit of targeted drug core genes and tumor-related genes and improve sensitivity;

S2, obtain the patient's genome mutation signal, pre-process the WDC sequencing data obtained in S1 and align it with the hg19 human reference genome, obtain the DNA mutation signal of the tumor tissue sample and the DNA mutation signal of the blood cell sample, compare and retain the DNA mutation signal that only exists in the tumor tissue sample as the genome mutation signal, the DNA mutation signal includes one or more of somatic variation (SNV), insertion and deletion (Indel), fusion or other types of mutation;

S3, screen the tracking mutation signals, sort the genome mutation signals in S2 according to function and credibility, screen a preset number of genome mutation signals with the highest ranking as tracking mutation signals, and the sorting rules are as follows: first, driver mutations with important functions are given the highest ranking priority; secondly, they are sorted by mutation frequency and primary clone-subclone. For mutations with a mutation frequency greater than 5%, they are sorted from large to small according to mutation frequency; for mutations with a mutation frequency between 1% and 5%, they are sorted first by primary clone>subclone, and then by mutation frequency;

S4, prepare a personalized combination panel (CCP probe), design a tracking mutation signal sequence probe (customized probe) based on the tracking mutation signal, and mix it with the fixed mutation signal sequence probe (core probe) and SNP probe to prepare a personalized combination panel, where the fixed mutation signal sequence probe (core probe) is used to detect tumor evolution or second primary, and the SNP probe is used to identify the source of the sample and evaluate the degree of sample contamination;

S5, obtain personalized combined panel sequencing data of patient tumor tissue sample DNA, blood cell sample DNA and plasma cfDNA, construct a plasma cfDNA library containing UMI connectors, and mix different sample type libraries of tumor tissue sample DNA library, blood cell sample DNA library and plasma cfDNA library according to the mass ratio of 2:1:(6˜12); obtain the captured DNA library through CCP probe hybridization capture, sequence the captured DNA library, and obtain personalized combined panel sequencing data of tumor patients. By mixing with this mass ratio, the data volume of tumor tissue sample DNA, blood cell sample DNA and plasma cfDNA 1:1:(3˜6) can be obtained, which can balance the sequencing depth and cost at the same time. While achieving an ultra-high depth of 100,000× for plasma, the tissue can reach a depth of 10,000× to obtain a more accurate tissue mutation spectrum, and the depth of more than 10,000× for blood cells can assist plasma in eliminating the interference of clonal hematopoiesis;

S6, track mutation signal correction and determine the tracking mutation sequence and position, utilize personalized combined panel sequencing data from tumor tissue samples and blood cell samples to rectify tracking mutation signals; eliminate signals that are no longer considered to be somatic small mutations and fusion mutations; remove mutations of clonal hematopoietic origin; update the tracking mutation signals to generate the final tracking mutation signals and ascertain the final sequence and position of the tracking mutation signals.

S7, obtain the tracking mutation signal detection results of plasma cfDNA, extract the reads pairs of plasma samples covering the final tracking mutation signal position, extract the molecular tag sequences at both ends, the starting position on the genome, the length and direction of the inserted fragment and other information, determine the single-strand consensus sequence (SSCS) and double-strand consensus sequence, and filter and determine the tracking mutation signal detection results in combination with the UMI sequence;

S8, combine the detection results of all tracking mutation signals to obtain the MRD detection results of the tumor patient, count the number of positive mutations of the tracking mutation signals in S7, and compare it with a preset threshold, if the count exceeds the threshold, the MRD status of the tumor patient is deemed positive; otherwise, it is negative.

Furthermore, the genes in the targeted drug gene panel in the above S1 include one or more genes from AKT1, ALK, AR, ARAF, BRAF, BRCA1, BRCA2, CDK4, CTNNB1, DDR2, EGFR, ERBB2, ERBB3, ERRFI1, ESR1, FBXW7, FGFR1, FGFR2, FGFR3, FLT1, GNA11, GNAQ, HRAS, IDH1, IDH2, KIT, KRAS, MAP2K1, MAPK1, MET, MTOR, NF1, NF2, NOTCH1, NRAS, NTRK1, NTRK2, NTRK3, PDGFRA, PIK3CA, PTEN, and RA One or more of the following genes: C1, RB1, RET, RICTOR, ROS1, SMAD4, TERT, TP53, TSC1, VEGFA, AKT2, AKT3, APC, ATM, ATR, ATRX, CDK6, CDKN2A, CHEK2, FLT3, FLT4, JAK1, JAK2, KDR, KEAP1, MDM2, MYC, PALB2, VHL, ABL1, BTK, SMO, ETV6, EWSR1, NTRK, HER2 and BRCA. The indications include one or more of solid tumors such as lung cancer, colorectal cancer, breast cancer, gastric cancer, gastrointestinal stromal tumor, thyroid cancer, head and neck squamous cell carcinoma, ovarian cancer and melanoma. The genetic status of a tumor, especially the mutation status of tumor driver genes, can indicate tumor progression, drug allergy or resistance, and can also be used to assess prognosis, recurrence, and metastasis risk. The panel composed of such genes is a targeted drug gene panel. Furthermore, different target genes or combinations can be selected as needed.

Furthermore, in the above S1, the WES probe and the targeted drug gene panel are mixed in a ratio of 1:2, and the WES other regions:tumor-related gene regions:targeted drug core gene regions can achieve an effective depth ratio of 1:1.5:2 after deduplication.

Furthermore, in the above S1, the WES probe and the targeted drug gene panel are mixed in a 1:4 ratio, and the WES other regions:tumor-related gene regions:targeted drug core gene regions can achieve an effective depth ratio of 1:2:3 after deduplication.

Furthermore, in the above S1, the WES probe and the targeted drug gene panel are mixed in a ratio of 1:8, and the WES other regions:tumor-related gene regions:targeted drug core gene regions can achieve an effective depth ratio of 1:3:6 after deduplication.

Furthermore, in the above S1, the tumor tissue sample may be a separated formalin-fixed and paraffin-embedded tumor tissue sample.

Furthermore, in S2 above, WDC sequencing data preprocessing includes removing adapters and low-quality bases, and the use of Trimmomatic software is recommended.

Furthermore, in S2 above, it is recommended to use BWA software for alignment to the hg19 human reference genome sequence.

Furthermore, in the above S2, after alignment to the hg19 human reference genome sequence, it also includes deduplication, realignment and quality value correction. Deduplication includes calling the commercial software Sentieon-202112.05, and using the command “sentieon driver—algo Dedup—rmdup” to deduplicate the initial Bam file to generate a deduplicated Bam file; realignment includes calling the commercial software Sentieon-202112.05, and using the command “sentieon driver—algo Realigner” to realign the deduplicated Bam file to generate a realigned Bam file; quality value correction includes calling the commercial software Sentieon-202112.05, and using the command “sentieon driver—algo QualCal” to perform quality value correction on the realigned Bam file to generate a corrected Bam file.

Furthermore, in the above S2, somatic variation (SNV) detection includes obtaining an initial somatic mutation list by comparing the corrected Bam files of the tumor tissue sample and the blood cell sample.

Furthermore, in the above S2, the fusion mutation detection includes obtaining the fusion mutation detection result of the tumor tissue sample by comparing the corrected Bam files of the tumor tissue sample and the blood cell sample.

Furthermore, in the above S2, the corrected data of the tumor tissue sample and the blood cell sample are compared, and the somatic mutations and fusion mutations of the patient to be tested are found using a pairing method. It is recommended to use Mutect2 software.

Furthermore, in the above S2, the genomic mutation signal also includes filtering, and the filtering rules are as follows: the population mutation frequency of the three databases, gnomAD, ExAC, and 1000 g, is less than 2%; the sequencing depth is greater than 40; the mutation frequency is greater than 1%; and it is not in the platform blacklist range (through statistics of a large number of samples and different batches, recurring low-quality mutations are defined as blacklist mutations).

Furthermore, in the above S2, the genome mutation signal filtering rules also include: support reads>2, coverage depth>100, no significant difference in positive and negative chain support, no simple repetitive sequences in and around, and tumor tissue mutation frequency/blood cell mutation frequency>5.

Furthermore, in the above S2, other tumor-related detection information of the patient can also be provided, including TMB, MSI, etc.

Furthermore, in the above S3, the classification of main clones and subclones is based on the genome mutation signals and CNV detection results in S2, the number of supporting mutation reads and sequencing depth of each somatic mutation, and considering the allelic imbalance introduced by CNV, etc., using statistical clustering methods, such as Bayesian clustering methods, to estimate the tumor purity and group somatic mutations into different clone groups, and count the cell proportion of each clone group, define the clone group with the highest proportion as the main clone, and define other categories as subclones. Furthermore, it is recommended to use factes and pyclone software to complete the classification.

Furthermore, the CNV detection includes obtaining an estimated value of the tumor purity of the tumor tissue sample and the tumor cell allele copy number by comparing the corrected Bam files of the tumor tissue sample and the blood cell sample.

Furthermore, in the above S3, the preset number is 10 to 50 or all mutation signals.

Furthermore, in the above S4, the design rules of the tracking mutation signal sequence probe (customized probe) are as follows: if it is an SNV/Indel type mutation, according to the reference genome and the tracking mutation list, the three sequences of the reference genome sequence 60 bp upstream of the starting position of each tracking mutation signal, the tracking mutation signal sequence and the reference genome sequence 60 bp downstream of the ending position of the tracking mutation signal are concatenated as candidate customized probe sequences; if it is a Fusion type mutation, according to the reference genome and the direction of the fusion mutation, the sequence of 60 bp upstream (along the transcript direction) of the breakpoint 1 of the upstream gene gene1 of the fusion mutation and the sequence of 60 bp downstream (along the transcript direction) of the breakpoint 2 of the downstream gene gene2 of the fusion mutation are concatenated as candidate customized probe sequences.

Furthermore, in the above S4, the design of tracking mutation signal sequence probes also includes filtering, and the filtering rules are as follows: remove candidate probe sequences with more than 20 “better matching positions” in the entire reference genome, where “better matching positions” refer to positions with a matching length greater than 30 bp and a matching expectation value less than 0.000001; remove candidate probe sequences containing repetitive sequence SSRs; remove abnormal candidate sequences with GC 80%.

Furthermore, in the above S4, the fixed mutation signals (high evidence hotspots) in the Core probe include evidence loci from NCCN guidelines, expert consensus, targeted evidence loci and chemotherapy resistance evidence loci in public databases, FDA/NMPA drug labels, combined clinical trials and conference abstracts, and at the same time, one or more of the sets formed by first-level evidence loci and second-level evidence loci are screened out in multiple cancer types.

Furthermore, in the above S4, the sites of the SNP probes include one or more of the SNPs site sets with higher heterozygosity in the dbSNP database covered by the whole exome in the WDC.

Furthermore, in the above S4, the genes of the fixed mutation signal sequence probes (core probes) are shown in Table 2, and the SNP probe coordinates are shown in Table 3.

Furthermore, in the above S4, the personalized panel is mixed according to the molar ratio of probe substances, Customized probe:Core probe:SNP probe=8:8:1, to prepare the CCP hybridization probe working solution, which is formulated according to 8:8:1. It can achieve an effective depth ratio of 5:5:1 after deduplication, which can reduce the detection limit of core genes/tumor-related genes for targeted medication and improve sensitivity.

Furthermore, in the above S5, the tumor tissue sample DNA library, the blood cell sample DNA library and the plasma cfDNA library are mixed in a mass ratio of 2:1:6 to obtain a data volume of tumor tissue sample DNA, blood cell sample DNA and plasma cfDNA of 1:1:3.

Furthermore, in the above S5, the tumor tissue sample DNA library, the blood cell sample DNA library and the plasma cfDNA library are mixed in a mass ratio of 2:1:9 to obtain a data volume of 1:1:4 for the tumor tissue sample DNA, the blood cell sample DNA and the plasma cfDNA.

Furthermore, in the above S5, the tumor tissue sample DNA library, the blood cell sample DNA library and the plasma cfDNA library are mixed in a mass ratio of 2:1:12 to obtain a data volume of tumor tissue sample DNA, blood cell sample DNA and plasma cfDNA of 1:1:6.

Furthermore, in the above S5, after hybridization capture is completed, elution is performed by using a volume gradient increasing elution method, which can obtain higher target ratio data compared with conventional equal volume elution. After hybridization capture is completed, off-target reads in the system or adsorbed on the tube wall need to be cleaned away. Conventional operation steps all use the same volume of cleaning solution for cleaning. This application tests that the gradient volume increase method can effectively increase the cleaning of off-target reads adsorbed on the tube wall during the previous step of blowing or swirling cleaning, ultimately presenting a higher target ratio than conventional operations, and achieving a higher depth and corresponding detection sensitivity.

Furthermore, in the above S5, after the hybridization capture is completed, it is washed with 100 μL preheated washing buffer I, 145 μL preheated Stringent washing buffer I, 150 μL preheated Stringent washing buffer I, 50 μL+100 μL washing buffer I, 155 μL washing buffer II, and 160 μL washing buffer III in a gradient of increasing volumes to obtain the captured library.

Furthermore, in the above S6, the tracking mutation signal correction includes: processing the personalized combination panel sequencing data with reference to S2 and S3, obtaining a new tracking mutation signal, and matching whether the tracking mutation signal in S3 is in the new tracking mutation signal, deleting the mutation signal that does not exist in the new tracking mutation signal, and generating a final tracking mutation signal.

Furthermore, in the above S6, determining the final tracking mutation sequence and position includes: obtaining an extended mutant sequence, and according to the reference genome and the final tracking mutation signal, for each tracking mutation sequence, concatenating three sequences of the reference genome sequence with a length of a bp from its starting position to the upstream of the genome, the tracking mutation sequence and its ending position to the reference genome sequence with a length of a bp from the downstream of the genome as candidate sequences; if the candidate sequence can only be uniquely matched within the range of b bp upstream and downstream of the candidate sequence, then retain the candidate sequence as the tracking mutation sequence, and define the genome starting position of the concatenated sequence as the genome starting position of the tracking mutation sequence, and the genome ending position of the concatenated sequence as the genome ending position of the tracking mutation sequence; if the retention standard is not met, then increase the length by 1 bp, that is, (a+1) bp to start re-extending the upstream and downstream sequences and repeat the operation until the retention standard is met or the length of the concatenated sequence exceeds c bp.

Furthermore, the above-mentioned a is 3-4, b is 100-200, and c is 30-35. Furthermore, in S6, a is 3, b is 200, and c is 35.

Furthermore, in the above S7, a pair of reads with the same read ID number is marked as a fragment, and the fragment information is extracted: including the molecular tag sequences at both ends, the starting position on the genome, the length and direction of the inserted fragment, etc.

Furthermore, in the above S7, determining the single-stranded consensus sequence (SSCS) includes: taking the fragments with matching fragment information as a group, wherein the matching fragment information refers to the UMI sequence, the starting position or the difference of the inserted fragment within the error range of d bp, etc., and having almost completely identical fragment information; starting from the base position on the fragment corresponding to the genome starting position of the final tracking mutation signal sequence, to the base position on the fragment corresponding to the genome ending position of the tracking mutation sequence, comparing the number of each base type at each position base by base, and the base types include A, T, C, and G; determining the SSCS, if. Bₘₐₓ/B_{second}>f is satisfied, the base type of the SSCS at the position is the base type with the largest number, and the base type of the negative consensus sequence at the position is marked as N, where Bₘₐₓ represents the number of the base type with the largest number, and B_{second} represents the number of the base type with the second largest number.

Furthermore, d is 1.

Furthermore, f is 2.

Furthermore, in the above S7, the UMI sequence is combined to filter and determine the tracking mutation signal detection result, including: for each tracking mutation, the SSCS that completely matches the tracking mutation sequence is defined as a simplex, and two simplexes with paired molecular tag sequences are defined as a duplex (double-strand consistency); the tracking mutation is filtered and determined according to the following rules: if the smaller value of the distance between the edge of the tracking mutation on the simplex and the edge of the fragment is less than a preset threshold (j), or the number of bases on the simplex that are different from the reference genome sequence is greater than a preset threshold (n), then the simplex is defined as a low-quality simplex; the proportion of low-quality simplexes for each tracking mutation is counted, and if it is greater than a preset threshold (r), the mutation is considered to be a low-confidence mutation and is removed in subsequent analysis; the number of simplexes and duplexes of each tracking mutation after filtering is counted, and if the number of simplexes is greater than a preset threshold (s) and the number of duplexes is greater than a preset threshold (h), then the mutation is reported as a positive mutation.

Furthermore, the above j is 5.

Furthermore, the above n is 5.

Furthermore, the above r is 0.5.

Furthermore, the above s is 0.

Furthermore, h is 1.

Furthermore, in the above S8, the preset threshold is 1-3, and it can also be set as needed. Furthermore, the preset threshold is 1.

As the second aspect of the current application, it provides a detection device for MRD lesions, comprising:

A data input module, used to input WDC sequencing data of a patient's tumor tissue sample and preoperative blood cell sample, and input personalized combined panel sequencing data of the patient's tumor tissue sample, blood cell sample and plasma;

A data processing module, used to complete the acquisition of genomic mutation signals, screening of tracking mutation signals, correction of tracking mutation signals, determination of tracking mutation sequences and positions, and acquisition of tracking mutation signal detection results of plasma cfDNA according to the first aspect;

A result output module, used to output the MRD detection results of the tumor patient described in the first aspect.

As the third aspect of the current application, it provides an electronic device, comprising: one or more processors; a storage device on which one or more programs are stored, and when the one or more programs are executed by one or more processors, the one or more processors implement the method described in any implementation method of the above-mentioned first aspect.

As the fourth aspect of the current application, it provides a computer storage medium on which a computer program is stored, wherein when the program is executed by a processor, the method described in any implementation manner of the above-mentioned first aspect is implemented.

### 3. Beneficial Effects

Compared with the prior art, the current application has the following beneficial effects:
(1) The method for detecting MRD lesions provided in the current application uses a WDC combined sequencing method, namely, a differentiated depth WES+targeted drug gene panel. On the one hand, it includes whole exome sequencing. Compared with other single fixed panels, the differentiated depth whole exome/targeted drug gene panel can screen patient-specific mutation spectra in a larger range, significantly increase the number of traceable sites, and improve detection sensitivity; on the other hand, it includes a high-depth fixed enhanced panel method, which focuses on detecting areas with high frequency and clinical evidence in the general tumor patient database, and can detect more and lower-frequency tissue variation sites with high tumor frequency/high tumor evidence, solving the problem that low-frequency sites may be missed when conventional WES is used to detect tissue sample sites in the prior art, and can also include classic fusion intervals, which are usually not in the exon region; finally, the current application method can simultaneously provide other tumor marker indicators, such as TMB and MSI, etc. These indicators may perform better on whole exome sequencing (TMB) or on a high-depth fixed enhanced panel (MSI).
(2) The method for detecting MRD lesions provided in the current application can obtain more accurate detection results under limited detection cost control by screening a limited number of mutation signals as tracking mutation signals in a ranking manner based on function and credibility. Driver mutations, high-frequency mutations, and major clone mutations are all mutations that have a greater probability of being released into the plasma. By sorting them in this way, mutation signals that are more likely to be detected in plasma can be selected, thereby improving detection sensitivity.
(3) The method for detecting MRD lesions provided in the current application uses a personalized combination panel (CCP probe), i.e., a combination panel of 100,000× ultra-high depth personalized Customized probes+high-evidence/high-frequency hotspot Core probes+SNP probes. The use of mutant customized sequence probes can more efficiently capture the mutation signal of the sample to be tested. The fixed core sequence probe can prompt the user of the current application of important tumor evolution/the emergence of the second primary mutation. The fixed SNP sequence probe is used for quality control to distinguish whether the sample to be tested is contaminated. Compared with the existing amplicon method that is prone to contamination during dozens of cycles of amplification, the method of the current application can monitor unqualified samples caused by contamination and avoid the occurrence of false positives or false negatives. In other words, the method for detecting tiny residual lesions provided in the current application can not only monitor the mutation sites of tumor origin, but also simultaneously detect the second primary mutation sites and monitor tumor evolution, further improving the detection sensitivity and overcoming the limitation of the prior art of only tracking tissue mutation spectra.
(4) The method for detecting microresidual lesions provided in this application obtains 100,000× plasma ultra-high depth personalized combined panel data captured by tumor tissue sample DNA, blood cell sample DNA and plasma sample DNA, and uses it to update the tracking mutation list to improve the accuracy of tracking site variation detection. That is, by obtaining the DNA data of tumor tissue samples again through a high-depth personalized combination panel, it is possible to check whether the mutations determined using the WDC combination sequencing method are real mutations, reduce the situation where the tracked mutations are not real patient-specific mutations due to the sequencing depth limitation of the WDC combination sequencing method, and improve the accuracy of the test results.
(5) The method for detecting MRD lesions provided in the current application, when detecting the results of tracking mutation signals in the plasma sample to be tested, only uses the duplex information of the unique molecular identifiers (UMI) and a strict credibility filtering model to detect the reads covering the tracking site, and removes duplicate sequences through the unique molecular tags to improve the accuracy of single-point detection of plasma free ctDNA, thereby solving the problem that the data in the prior art cannot be accurately removed from duplicates; only detecting the reads covering the tracking site effectively reduces the computing cost compared to the variation detection of the entire interval; combining the duplex information of the molecular tag technology with a strict credibility filtering model, using an iterative method to find the unique matching extended mutant sequence can effectively improve the accuracy of Indel detection, and at the same time, using duplex and subsequent strict filtering models improves the accuracy of detection of various mutation types such as SNV, Indel and fusion.
(6) The method for detecting MRD lesions provided in this application is based on differentiated deep whole exome/targeted drug sequencing and tissue, blood cell, and plasma co-capture technology, and 100,000× ultra-high depth personalized/high evidence hotspot combination panel sequencing. It is a method for evaluating MRD lesions and tumor evolution/second primary in plasma samples. It overcomes the problems of existing methods such as high tissue detection limits or too few tracking sites, insufficient detection sensitivity and accuracy, or high detection costs when the ctDNA content in the blood is low, and the inability to achieve both personalized tracking detection and tumor evolution/second primary detection. It significantly improves the accuracy of predicting the risk of recurrence after treatment for patients within a limited cost range.

### BRIEF DESCRIPTION OF THE DRAWINGS

FIG. 1 shows the number of mutations that can be tracked in Example 1 and Comparative Example 1.

FIG. 2 shows the positive mutations detected in Example 1 and Comparative Example 1.

FIG. 3 shows the differential sequencing depth of the WDC probe formed by mixing the whole exome sequencing probe and the targeted drug gene panel in different proportions.

FIG. 4 shows the sequencing data depth of CCP probe hybridization co-capture of tissue sample DNA libraries, blood cell sample DNA libraries and plasma cfDNA libraries with different mass ratios.

FIG. 5 is a comparison of the effects of medium volume washing and volume gradient washing in the hybrid capture system.

### DESCRIPTION OF THE EMBODIMENTS

The current application is further described below in conjunction with specific examples.

It should be noted that the terms such as “upper”, “lower”, “left”, “right”, “middle”, etc. cited in this specification are only for the convenience of description and are not used to limit the scope of implementation. Changes or adjustments to their relative relationships should be regarded as the scope of implementation of this application without substantially changing the technical content.

Unless otherwise defined, all technical and scientific terms used herein have the same meaning as commonly understood by those skilled in the art to which this application belongs; the term “and/or” used herein includes any and all combinations of one or more of the associated listed items.

If no specific conditions are specified in the examples, the experiments were carried out according to conventional conditions or conditions recommended by the manufacturer. The reagents or instruments used without indicating the manufacturer are all conventional products that can be purchased from the market.

As used herein, the term “about” is used to provide flexibility and imprecision associated with a given term, measurement, or value. The degree of flexibility for a particular variable can be readily determined by one skilled in the art.

As used herein, the term “at least one of” is intended to be synonymous with “one or more of.” For example, “at least one of A, B, and C” explicitly includes only A, only B, only C, and combinations of each thereof.

Concentrations, amounts, and other numerical data may be presented herein in a range format. It should be understood that such range format is used merely for convenience and brevity and should be interpreted flexibly to include not only the values expressly recited as the limits of the range, but also to include all individual values or sub-ranges within the range, as if each value and sub-range were expressly recited. For example, a numerical range of about 1 to about 4.5 should be interpreted to include not only the explicitly recited limit of 1 to about 4.5, but also include individual numbers (such as 2, 3, 4) and sub-ranges (such as 1 to 3, 2 to 4, etc.). The same principle applies to ranges reciting only one numerical value, such as “less than about 4.5”, which should be interpreted to include all the aforementioned values and ranges. Furthermore, this interpretation should apply regardless of the breadth of the scope or features being described.

### Example 1

This example detects MRD in the preoperative plasma of 51 patients with stage I lung cancer. Since the plasma is preoperative plasma, it can be understood that the above plasma samples are MRD positive samples, including the following steps:

S1: Obtain WDC sequencing data of the patient's tumor tissue DNA and blood cell DNA, namely: construct a tumor tissue DNA library and a blood cell DNA library respectively; mix the two libraries in equal mass ratio, and use WDC probes for hybridization capture to obtain a captured DNA library, wherein the WDC probe is a mixed probe formed by mixing the whole exome sequencing probe (WES probe) with the targeted drug gene panel in a ratio of 1:(2˜8); sequence the captured DNA library to obtain the WDC sequencing data of the tumor patient. The specific steps include:

S11: DNA extraction and nucleic acid fragmentation. Tumor tissue samples and preoperative whole blood were collected from patients. Blood cell samples and plasma samples were obtained by density gradient centrifugation. DNA from tumor tissue samples was extracted and diluted to 0.5 ng/μL˜6 ng/μL. DNA from blood cell samples was extracted and diluted to 6 ng/μL. cfDNA in plasma was extracted and diluted to 0.5 ng/μL˜1 ng/μL. The tumor tissue sample DNA and blood cell sample DNA were processed using a nucleic acid fragmentor to obtain fragmented tumor tissue sample DNA and fragmented blood cell sample DNA. In an example, the tumor tissue can be an isolated formalin-fixed, paraffin-embedded tumor tissue sample.

S12: Construction of DNA libraries of tumor tissue samples and blood cell samples. The fragmented tumor tissue sample DNA and fragmented blood cell sample DNA were end-repaired and A-added using Roche's KAPA Hyper Prep kit (KK8504). The pre-amplification reaction was performed using Roche's KAPA HiFi HotStart ReadyMix (KK2602) kit. The pre-amplification products were purified into new EP tubes using Beckman's AMPure XP beads to obtain the DNA libraries of tumor tissue samples and blood cell samples. In the example, the DNA library can also be subjected to Qubit concentration detection and Agilent 2100 quality inspection, and the nucleic acid concentration detector is used to quantify the tumor tissue sample DNA library ≥800 ng and the blood cell sample DNA library ≥500 ng; and the library is analyzed by a bioanalyzer, and the main peaks of the tumor tissue sample and blood cell sample DNA libraries should be between 150 and 500 bp.

S13: WDC probe hybridization capture obtains the captured DNA library (WDC library), and the target region fragments are captured using the WDC probe to construct the captured DNA library. In the example, the WDC probe is a mixed probe formed by mixing the WES probe and the targeted drug gene panel at a ratio of 1:(2˜8). The probes mixed in this ratio can achieve differentiation in sequencing depth, that is, WES other regions:tumor-related gene regions:targeted drug gene regions can achieve an effective depth ratio of 1:(1.5˜3):(2˜6), which can reduce the detection limit of targeted drug genes and tumor-related genes and improve sensitivity. In an example, the genes targeted for drug use include AKT1, ALK, AR, ARAF, BRAF, BRCA1, BRCA2, CDK4, CTNNB1, DDR2, EGFR, ERBB2, ERBB3, ERRFI1, ESR1, FBXW7, FGFR1, FGFR2, FGFR3, FLT1, GNA11, GNAQ, HRAS, IDH1, IDH2, KIT, KRAS, MAP2K1, MAPK1, MET, MTOR, NF1, NF2, NOTCH1, NRAS, NTRK1, NTRK 2. NTRK3, PDGFRA, PIK3CA, PTEN, RAC1, RB1, RET, RICTOR, ROS1, SMAD4, TERT, TP53, TSC1, VEGFA, AKT2, AKT3, APC, ATM, ATR, ATRX, CDK6, CDKN2A, CHEK2, FLT3, FLT4, JAK1, JAK2, KDR, KEAP1, MDM2, MYC, PALB2, VHL, ABL1, BTK, SMO, ETV6, EWSR1, NTRK, HER2 and BRCA. In the example, the WDC library is constructed as follows: the tumor tissue sample DNA library and the blood cell sample DNA library are mixed in equal mass ratios according to the sample type, and placed in a vacuum centrifugal concentrator for evaporation at 60° C. for about 20 min to obtain an evaporated library; the DNA hybridization system and the WDC hybridization probe are added to the evaporated DNA library, and the mixture is incubated at room temperature after oscillation and centrifugation, and hybridization is performed according to the hybridization reaction conditions of 95° C. for 30 s and 70° C. for 16 hours; the hybridized library is subjected to target region hybridization capture and post-hybridization elution using the commercially available kit Twist Standard Hyb and Wash Kit (104447), and the beads with target region fragments after elution are subjected to post-hybridization amplification reaction using the KAPA HiFi HotStart ReadyMix (KK2602) kit, and finally the pre-amplification product is purified into a new EP tube using Beckman's AMPure XP beads, which is the DNA library after WDC probe hybridization capture (WDC library). In an example, Qubit concentration detection can also be performed on the DNA library. In the example, the commercially available kit xGen™ Hybridization and Wash Kit (1080584) can also be used to perform hybridization capture of the target region and post-hybridization elution to achieve the same effect.

S14: Sequencing of WDC library to obtain WDC sequencing data. In the example, specifically: the WDC library is sequenced on a gene sequencer to obtain a data output of 10:3 for tumor tissue samples and blood cell samples.

S2: Obtain the patient's genome mutation signal, that is, pre-process the WDC sequencing data obtained in S1 and compare it with the hg19 human reference genome to obtain the DNA mutation signal of the tumor tissue sample and the DNA mutation signal of the blood cell sample, and retain the DNA mutation signal that only exists in the tumor tissue sample as the genome mutation signal. The DNA mutation signal includes one or more of somatic variation (SNV), insertion and deletion (Indel), fusion or other types of mutations. The specific steps include:

S21: WDC sequencing data preprocessing and alignment, including removal of adapters and low-quality bases, alignment to the hg19 human reference genome sequence, deduplication, re-alignment and quality value correction to obtain the corrected Bam file. In an example, WDC sequencing data pre-processing is performed using commercial software. In an example, removing adapters and low-quality bases includes calling Trimmomatic-0.36 to treat each pair of FASTQ files as paired reads to remove adapters and low-quality bases, and using the “ILLUMINACLIP:TruSeq3-PE.fa:2:30:10:8:true LEADING:3 TRAILING:3 SLIDINGWINDOW:4:20 MINLEN:51” parameters to generate a FASTQ file after removing the adapter. In an example, aligning to the hg19 human reference genome sequence includes calling the commercial software Sentieon-202112.05 to use the FASTQ file after removing the connector as paired reads, using the bwa men module to align to the hg 19 human reference genome sequence, and using the util sort module to sort the alignment results to generate an initial Bam file. In an example, deduplication includes calling the commercial software Sentieon-202112.05, using the command “sentieon driver—algo Dedup—rmdup” to deduplicate the initial Bam file, and generating a deduplicated Bam file. In an example, the re-alignment includes calling the commercial software Sentieon-202112.05, using the command “sentieon driver—algo Realigner” to re-align the deduplicated Bam file to generate a re-aligned Bam file. In an example, the quality value correction includes calling the commercial software Sentieon-202112.05, using the command “sentieon driver—algo QualCal” to perform quality value correction processing on the Bam file after re-alignment, and generating a corrected Bam file.

S22: Somatic variation (SNV) detection, including obtaining an initial list of somatic mutations by comparing the corrected Bam files of tumor tissue samples and blood cell samples. In an example, the comparison is performed by processing the corrected Bam file using commercial software. In the example, the paired sample mode of the Mutect2 module of gatk-package-4.1.9.0 is called to obtain an initial somatic mutation list. In the example, the FilterMutectCalls module of gatk-package-4.1.9.0 is used to filter out mutations whose certain indicators do not meet the default conditions of the software, and the specific indicators include: map_qual, base_qual, germline, fragment, normal_artifact, position and haplotype. In an example, mutation annotation is also included to obtain site information for subsequent site filtering and sorting operations. In an example, mutation annotation is performed by commercial software. In the example, the initial mutation list is annotated using ANNOVAR software to generate an annotated mutation list, using the parameters: -protocol refGene, ljb26_sift, ljb2_pp2hdiv, ljb2_pp2hvar, exac03, clinvar_20220709, cadd14, gnomad_exome, cytoBand, snp138, gnomad_genome, 1000g2015aug_all, 1000g2015aug_chb, 1000g2015aug_ch s, 1000g2015aug_afr, 1000g2015aug_eas, 1000g2015aug_eur, 1000g2015aug_sas, 1000g2015aug_a mr, simpleRepeat, cosmic80, HGMD, rmsk, BIC, OMIM, reliability, Pro_CancerRepeat, hgmd_202004.

S23: Fusion mutation detection, including obtaining the fusion mutation detection results of tumor tissue samples by comparing the corrected Bam files of tumor tissue samples and blood cell samples. In an example, the comparison is performed using commercial software to process the corrected Bam files. In the example, LUMPY (V0.2.13) software is called, and the corrected Bam files of paired tumor tissue samples and blood cell samples are input to obtain the fusion mutation detection results of the tumor tissue samples.

S24: Copy number variation (CNV) detection, including obtaining the estimated values of tumor purity of tumor tissue samples and tumor cell allele copy number by comparing the corrected Bam files of tumor tissue samples and blood cell samples. In an example, the comparison is performed by processing the corrected Bam files through commercial software. In the example, the R package FACTES is called, and the paired tumor tissue samples and the corrected Bam files of the blood cell samples are input to obtain the estimated values of the tumor purity of the tumor tissue samples and the copy number of the tumor cell alleles, which are used for the subsequent classification of the main clones and subclones.

In the example, S25 is also included: mutation filtering, including filtering out mutations according to the following filtering rules to obtain the final genome mutation signal, the filtering rules including: the population mutation frequency of the three databases of gnomAD, ExAC, and 1000 g is less than 2%; the sequencing depth is greater than 40; the mutation frequency is greater than 1%; it is not in the platform blacklist range (through a large number of samples, statistics of different batches, repeated low-quality mutations are defined as blacklist mutations); support reads>2; coverage depth>100; there is no significant difference in positive and negative chain support; there are no simple repetitive sequences in and around; tumor tissue mutation frequency/blood cell mutation frequency>5.

In the example, TMB and MSI analysis is also included. The analysis method refers to the Chinese invention patent with publication number CN112029861B, the invention name of which is “Tumor mutation load detection device and method based on capture sequencing technology” and the invention name of which is “Microsatellite sites for detecting MSI, screening methods and applications thereof” with publication number CN112365922B.

S3: Screening and tracking mutation signals, that is, sorting the genomic mutation signals in S2 according to function and credibility, firstly, giving the highest ranking priority to the driver mutations with important functions; secondly, sorting by mutation frequency and primary clone-subclone, and sorting from large to small by mutation frequency for mutations with mutation frequency greater than 5%; for mutations with mutation frequency between 1% and 5%, sorting by primary clone>subclone is preferred, and sorting by mutation frequency is second priority, and after sorting, a preset number of genomic mutation signals with the highest ranking are screened as tracking mutation signals, which specifically includes the following steps:

S31: Classification of main clones and subclones. According to the genomic mutation signals and CNV detection results in S2, the number of supporting mutation reads and sequencing depth of each somatic mutation, and considering the allelic imbalance introduced by CNV, etc., statistical clustering methods, such as Bayesian clustering methods, are used to estimate the tumor purity and group somatic mutations into different clonal populations, and the cell proportion of each clonal population is counted. The clonal population with the highest proportion is defined as the main clone, and the other categories are defined as subclones. In an example, the classification is performed by a commercial software process. In the example, the run_analysis_pipeline module of the PyClone-0.13.1 software is called, and the parameters “--num_iters 10000--burnin 1000--prior major_copy_number--max_clusters 2” are used to determine the classification of each mutation, that is, whether it belongs to the main clone or the subclone, according to the genomic mutation signal and the CNV detection result.

S32: Sorting: sorting is performed according to the following sorting rules: based on the pre-summarized driver mutation database with important functions, the mutations in the database are screened and given the highest sorting priority; sorting is performed by mutation frequency and primary clone-subclone. For mutations with a mutation frequency greater than 5%, they are sorted from large to small according to mutation frequency; for mutations with a mutation frequency between 1% and 5%, they are sorted according to primary clone>subclone first, and then sorted according to mutation frequency.

S33: Screening and tracking mutation signals, including selecting the genomic mutation signals ranked at the top in S32 as tracking mutation signals. In an example, the top 50 genomic mutation signals are selected as tracking mutation signals. In an example, all genomic mutation signals are selected as tracking mutation signals.

S4: Prepare a personalized combination panel (CCP probe working solution), that is, design a tracking mutation signal sequence probe (customized probe) according to the tracking mutation signal, and mix it with the fixed mutation signal sequence probe (core probe) and SNP probe to prepare a personalized combination panel, wherein the fixed mutation signal sequence probe (core probe) is used to detect tumor evolution or second primary, and the SNP probe is used to identify the source of the sample and evaluate the degree of sample contamination, which specifically includes the following steps:

S41: Screen candidate customized probe sequences, the screening rules are as follows: If it is an SNV/Indel type mutation, according to the reference genome and the tracking mutation signal, the three sequences of the reference genome sequence 60 bp upstream of the starting position of each tracking mutation signal sequence, the tracking mutation signal sequence, and the reference genome sequence 60 bp downstream of the ending position of the tracking mutation signal sequence are concatenated as candidate customized probe sequences; If it is a Fusion type mutation, according to the reference genome and the direction of the fusion mutation, the sequence of 60 bp upstream (along the transcript direction) of the breakpoint 1 of the upstream gene gene1of the fusion mutation and the sequence of 60 bp downstream (along the transcript direction) of the breakpoint 2 of the downstream gene gene2 of the fusion mutation are concatenated as candidate customized probe sequences. This method uses probe sequences targeting specific tracking mutation signals, which can more effectively capture sequences of specific tracking mutations and improve detection sensitivity. However, traditional probe sequences based on reference genomes will have a weakened ability to capture sequences of these specific tracking mutations due to the reduced matching between fragments of sequences carrying specific tracking mutations and probes.

S42, candidate customized probe sequence filtering, the filtering rules are as follows: remove candidate probe sequences with more than 20 “better alignment positions” in the entire reference genome, where “better alignment positions” refer to positions with a matching length greater than 30 bp and an alignment expectation value less than 0.000001; remove candidate probe sequences containing SSR; remove abnormal candidate sequences with GC 80%. In an example, the above filtering can be performed by commercial software. In an example, the blat (V.35) software is called to remove probe sequences having more than 20 “good alignment positions” in the entire reference genome. In an example, the software MISA is called to detect repeated sequences SSRs, and candidate sequences containing SSRs are removed. In the example, MFEprimer (v.3.2.6) software was called to perform quality control (GC, Tm and Dg) on the candidate probe sequences, and abnormal candidate sequences with GC 80% were removed.

S43, prepare CCP probe working solution, mix according to the probe molar number of Customized probe:Core probe:SNP probe=8:8:1, the Customized probe is shown in Table 1, the gene of the fixed mutation signal sequence probe (core probe) is shown in Table 2, and the coordinates of the SNP probe are shown in Table 3. The CCP probe prepared in 8:8:1 can achieve an effective depth ratio of 5:5:1 after sequencing, which can reduce the detection limit of targeted drug genes/tumor-related genes and improve sensitivity. The Core probe and SNP probe required for the preparation of CCP probe working solution have different functions. Since the Core probe needs to detect tumor evolution or second primary tumors, it also requires a plasma data depth of 100,000× to increase the detection sensitivity. The SNP probe is only used to identify the source of the sample and evaluate the degree of sample contamination, so it only requires a lower data depth. In the example, the Core probe comes from the Zhenhe Tumor Precision Medicine Evidence Library, in which the evidence gene loci are all from the NCCN guidelines, expert consensus, targeted evidence gene loci and chemotherapy resistance evidence gene loci in public databases, FDA/NMPA drug labels, combined with clinical trials and conference abstracts and other evidence gene loci. At the same time, primary evidence gene loci and secondary evidence gene loci are screened out from multiple cancer types, and the formed collection is a fixed mutation signal panel (core panel). In the examples, the SNP probe is used to identify the source of the sample and assess the degree of sample contamination, which is an indispensable part of ensuring the accuracy of sample detection. The SNP probes are mainly derived from the set of SNPs with higher heterozygosity in the dbSNP database covered by the whole exome in WDC. In the example, the Core probe, the SNP probe and the Customized probe are mixed according to the molar ratio of the probes, and the system also includes IDTE.

TABLE 1
Customized probes

| Probe ID | chr | seq start | seq end | seqs |
|---|---|---|---|---|
| P236943_P037 | chr12 | 56236088 | 56236207 | AAGACCTTGAGACCTTAGCCCTAAAGGCATACACCTCATAGCTTCTCACCTCAGAGCCTTG |
| | | | | GTGATATCTTCTGGCTTGAAGTTATTAGATCCTTCTAGAGGCTTCTGTAGGTACCCATA |
| P237607_P007 | chr5 | 176072378 | 176072497 | CTGCTGTGTCTGATCGGGGAGAGCTTTGAGGAACACAGCAGAGAGGTATGTGGGGCCGTTG |
| | | | | TCAACATCCGCACCAAGGGGGACAAGATCGCTGTGTGGACGAGGGAGGCGGAAAACCAG |
| P237607_P012 | chr8 | 2040129 | 2040248 | AAGCTCTGATGCCATTTCACCACTCCTTTTGTCTCCTACGAAAAGTTGTCCCTTCTGCTTC |
| | | | | GGGTCGGGTTCTTGCTTCCCGAAACACCAAGACGTCGGTGGTGGTGCAGTGGGACCGAC |
| P238684_P016 | chr17 | 73945313 | 73945432 | CGCGTTCTGACTGATTCCATACAGAGAATACAGCAGACATAAACTCCTTAAGACAGCTTAA |
| | | | | ATGGCTTTATCTTGAATTTTGAGGAGTTTTTCTGAAAAGAGCTTAACTACCACATAGTG |
| P238791_P020 | chr17 | 21102079 | 21102198 | TCAAACTGGTCTTGGGCATTCTCCCCATTGTAGATCTCATGCACAATGTAGCAGTCTGTAG |
| | | | | CCGACAAGCTCACCCTTTTGATGGAGGGGGCAGGAAAGGGAGAGAGAGAGAGAGACAGG |
| P238792_P001 | chr3 | 41266054 | 41266173 | CAGAAAAGCGGCTGTTAGTCACTGGCAGCAACAGTCTTACCTGGACTCTGGAATCCATTTT |
| | | | | GGTGCCACTACCACAGCTCCTTCTCTGAGTGGTAAAGGCAATCCTGAGGAAGAGGATGT |
| P238792_P048 | chr8 | 124195455 | 124195574 | GCTGGGCCTCAGCTGAGAAGCCCTACCTGAAGGAAAAATCCAGCGCCACTGTGTACTTCTA |
| | | | | GACCGTCAAGCACAACAACATCAGAGACCTCGTCCGCCGCTGCATCACCCGGACTAGCC |
| P239145_P042 | chr3 | 52256128 | 52256247 | TTGGCGCTAAGGTTGAGCTCTCGCAGCTCCTTGGCCTTGGAAAAGAAGCCGGGGGçCAçAA |
| | | | | AGCTGATGCTGTTGCAGCTGACATCCAGCCTCCGGAGCCGGGTGCCAGCAGGCAGGCTG |
| P239362_P030 | chr7 | I29765669 | 129765788 | TTTATGTATGTGTATATGTTCTTTTTTTTCTCCAAGATCCATGCATACAACCTTGAAACAA |
| | | | | ATGCCTGGGAGGAAATTGCAACAAAACCCCATGAAAAAATAGGTAAATTTAAAGTATTG |
| P239497_P019 | chr11 | 67185930 | 67186049 | CGTTCTGGGCTCCCCCAGCACCTTTCTGCCTGTGCTGCTGGAGGGTGGGGTCCAGAGCCTG |
| | | | | GGTGAGTGTATGCTCAAGTTTCCCCATCCCCTTCTACAGAAAGGGCAGCCCTGCCCTGG |
| P239643_P042 | chr8 | 27823986 | 27824105 | GACAGCATGCTTCAGGGCCGACAGGGACCCCAGCTGGGTACAGCAGATGCTTGCCCGCCAT |
| | | | | TTGTGACATGGACCTGGACAATAAGGGTAAGAGGAGGAAGGAAAGAAGACACTTAAAAA |
| P239729_P010 | chr10 | 120460847 | 120460966 | ACATGTTCCGTAAACAGCTTTATAAGGTCATCTTTTAAGTCTCTGTTAAGCTTGGTTTCAA |
| | | | | TGTAAAACTTATTCTGAAAAATAAAATAAAATCTTTTTTTGTGTATTAATTGGGGAAAT |
| P241180_P002 | chr12 | 49438166 | 49438285 | ACCCCATCCCAGGACCTCACCAGGCCGATATGGTTTACGCTTGCGTTTTTTGCTTTCCTTG |
| | | | | GTCTCCTCTTTGCCAGGCTCCACATCAGGGCTGACGGGGCCCTCCAGTTTAATTTCGCA |
| P241180_P017 | chr18 | 74580654 | 74580773 | CTGAGTGTGGGGATGAGTTTACTCTGCAGAGTCAGCTGGCCGTGCACATGGAGGAGCACTG |
| | | | | CCAGGAGCTGGCTGGAACCCGGCAGCATGCCTGCAAGGCCTGCAAGAAAGAGTTCGAGA |
| P243381_P020 | chr5 | 140230311 | 140230430 | CGGTGGGGAGTTGGTCGTACTCGCAGCAGAGGAGGCAGAGGGTGTGCTCTGGCGAGGGTCC |
| | | | | GCAGAAGACCGACCTCATGGCCTTCAGCCCGGGCCTTTCTCCTTGTGCTGGATCTACAG |
| P243381_P022 | chr15 | 88613034 | 88613153 | ATTTGCTGAACTCACAGAGGGAAGCTGGCTTGGTGAAAAGGCCAGGGATCTCAGGCCCCAA |
| | | | | ACAGCCCAGAAATACAAGGTGCCTCCTCTCTGGCACTTGGAAAGTCAGAACTCACAGGT |
| P243381_P029 | chr11 | 18158827 | 18158946 | GACCCTGAGCTTCACGGGGCTGACGTGCATCGTTTCCCTTGTCGCGCTGACAGGAAACGTG |
| | | | | GTTGTGCTCTGGCTCCTGGGCTGCCGCATGCGCAGGAACGCTGTCTCCATCTACATCCT |
| P243381_P034 | chr6 | 32362493 | 32362612 | ACCTGAAGGAACATAAGGAATCACCAACCTGAGAGAGAAAAAGTTGCGATTTTCTCCTCAC |
| | | | | CCAAAAAGGGGATGCTGATGGAACAAGTGACGTCCACAGCGGAGATGTTTGTGACCCTT |
| P243381_P035 | chr12 | 6752717 | 6752836 | ACCAAGGATCTCCTCCTCCATGTACTTCCAGGCCTTGGCTGTGGGGGTTATGATGCAGGCA |
| | | | | TTCTCCACGATCGAATAGCACAGCACCAGCAAGGCCTCTGTGTGGGGCAGCTGCAGGAG |
| P244057_P005 | chr17 | 78272168 | 78272287 | GGACGTACACCTGGCTGGGCGCCCTGCCTGTCCTGCACTGCTGTATGGAGCTGGCCCCGTG |
| | | | | GCACAAGGATGCCTGGAGACAGCCTGAGGACACCTGGGCCGCTCTGGAGGGACTCTCCT |
| P244057_P039 | chr9 | 87349766 | 87349885 | TAATACAGCCCTTTAAGGAGAAAATTAAGTCAAAAATTTGACCCACCTTTCAGAGGGTGTG |
| | | | | TCAGTCTTAGATCTTTCTGCTGTCTAAGCTCTTTTCCCCCTTCTCCTTTTCATTTGGAA |
| P244060_P003 | chr14 | 100380955 | 100381074 | TCTTGACCTGTGGGCATGACAAGCATGCCACTCTCTGGGACGCTGTGGGTCACCGTCCCAT |
| | | | | CTGGGACAAAATAATAGAGGTAAACATGCACATTACATTTCCATTTTTCTTACAGAAAT |
| P244060_P009 | chr1 | 150801630 | 150801749 | ATGTTGTGTCGGGAGATGAACTCTGTEGGTTGACAAACATTACTCATGTCTGTACAGTTAG |
| | | | | GAGAACTAGTTACCTGAGAGTGAAGAGATAAAAATGAGGTAAAATGATACCATTATTTA |
| P244060_P022 | chr9 | 136804213 | 136804332 | GTGACTCCTCACCTTTCCAAAGTCTCGCACATCGAAGAGGTCAAAGGGGTCAAACAGCTTG |
| | | | | CTGTTCCTTAATCCAAATTTATCGTGGCAGACTTTCAGGAAGGTGCGTATGTTCTTCAA |
| P269214_P002 | chr12 | 112926829 | 112926948 | TGTTGACTGCGATATTGACGTTCCCAAAACCATCCAGATGGTGCGGTCTCAGAGGTCAGTG |
| | | | | ATGGTCCAGACAGAAGCACAGTACCGATTTATCTATATGGCGGTCCAGCATTATATTGA |
| P269214_P032 | chr12 | 78401141 | 78401260 | GCACAGGAAATGGTGCTGTCCAACTCCCTCAACAGCAGCAACATAGCCACCCGAATACCAC |
| | | | | GACAGTGGCACCATTCATTTACAGGTAAGGTGGCCTCTGTTTATCCACAGTTGTAAATA |
| P269215_P012 | chr19 | 7142974 | 7143093 | CAAAAGGCCTGTGCTCCTCCGGACTCGTGGGCACGCTGGTCGAGGAAGTGTTGGGGAAAAC |
| | | | | TGCCACCGTGGGCACGGCCACCGTCACATTCCCAACATCGCCAAGGGACCTGCGTTTCC |
| P269215_P020 | chr12 | 20766395 | 20766514 | ACTGTGGACATCGCCGTCATGGGCGAGGCCCACGGCCTCATTACCGACCTCCTGGCAGAAC |
| | | | | CTTCTCTTCCACCAAACGTGTGCACATCCTTGAGAGCCGTGAGCAACTTGCTCAGCACA |
| P269228_P022 | chr2 | 160602295 | 160602414 | CATAGTCAAGTTCCTAGATCTTCATCAATGGTACTTGGATCATTTGGAACAGACTTAATAA |
| | | | | GAGAGAGGAGAGATTTGGAGAGAAGAACAGATTCCTCTATTAGTAATCTTATGGATTAT |
| P269231_P011 | chrX | 101911995 | 101912114 | TTCAAGCCTGGTCCATGGGGTAGGGTCGGCTTCCCATCTATAAGCCCCTTTAGATTTCCAA |
| | | | | AAGAGGCAGCATCTTTATTCTGTGAAATGTTTGGGGGCAAACCCAGGAACATGGTACTT |
| P269234_P007 | chr1 | 158622347 | 158622466 | CAGCATGTCTCCTGCCTCATAGGCCAATAAAAATTCATTATAACGTTGCAATAGACGACAT |
| | | | | CTGCGTTCTTCTGCCCGATCCAAGAGGGAGCGGTATCTGGATGGAGAATTGGGAAAAGT |
| P269237_P050 | chr6 | 161160151 | 161160270 | GAATCTCGAACCGCATGTTCAGGAAATAGAAGTGTCTAGGCTGTTCTTGGAGCCCACACAA |
| | | | | AAAGATATTGCCTTGCTAAAGCTAAGCAGGTACTCGTTCACCTGTGGTCTTCACCCCAC |
| P269238_P041 | chr20 | 54941267 | 54941386 | CGCTAGTCTCTGTGCCCTTGATTGTCAGATATTTTCGAAAAGTGGGATTTTTTAAACCTAC |
| | | | | AGCTGCAAAACCTTAATGAACTCTTCAGTCGTACACACTGAAAACCTATTTCTTCTAAA |
| P269243_P027 | chr7 | 120373034 | 120373153 | TTGCTCTACCTGTTCCGGTGATTGTATCCAACTTCAGTCGCATCTACCACCAGAATCAATG |
| | | | | AGCAGACAAACGAAGGGCACAAAAGGTGCGTATTCAACTCCGTGCAACCATGGTTTAGC |
| P269243_P029 | chrX | 153047206 | 153047325 | GCTTTGTGGCCCTCAAAGTGGTGAAGAGTGCGGGGCATTACACGGAGACAGCTGTGGATAA |
| | | | | GATCAAGCTCCTGAAATGTGTGAGGCACCTCCCTACCCCACTCCCAGCTCCCCTGGAGC |
| P269243_P037 | chr19 | 39230675 | 39230794 | AGTCCAGGTCTCTTACCAGGGCAGTGGCGCCCACGAGGGACTCCTTGGCCAGGGCATGGTG |
| | | | | CAGGGCAGAGAACAGCCCCATGCTGTTTTGTCTCAGATAGAGCACCTCGCCCACGCCGC |
| P269243_P042 | chr5 | 121362631 | 121362750 | TCCTAGATTTTCCACAGAATGAGCCTCAGATCAAGAATCAGTTTAATAAGAAGCTATCAAG |
| | | | | AAGACTTGAAAATACAAAACAGCAATTGCAGCTGCCTCTTCATCCTTCATGGGAAGCAA |

Customized probes in Table 1 are shown as SEQ ID NO: 1-SEQ ID NO:37 in order.

TABLE 2
Core probe genes

| SN. | Gene | Main transcript |
|---|---|---|
| 1 | AKT1 | NM_001014431 |
| 2 | ALK | NM_004304 |
| 3 | BRAF | NM_004333 |
| 4 | CTNNB1 | NM_001904 |
| 5 | EGFR | NM_005228 |
| 6 | ERBB2 | NM_004448 |
| 7 | ERBB3 | NM_001982 |
| 8 | ERBB4 | NM_005235 |
| 9 | ESR1 | NM_001122740 |
| 10 | FGFR1 | NM_023110 |
| 11 | FGFR2 | NM_000141 |
| 12 | FGFR3 | NM_000142 |
| 13 | FGFR4 | NM_213647 |
| 14 | HRAS | NM_005343 |
| 15 | IDH1 | NM_0058% |
| 16 | IDH2 | NM_002168 |
| 17 | KIT | NM_000222 |
| 18 | KRAS | NM_033360 |
| 19 | NRAS | NM_002524 |
| 20 | NTRK3 | NM_001012338 |
| 21 | PDGFRA | NM_006206 |
| 22 | PDGFRB | NM_002609 |
| 23 | PIK3CA | NM_006218 |
| 24 | RET | NM_020975 |
| 25 | ROS1 | NM_002944 |
| 26 | SMAD4 | NM_005359 |

TABLE 3
SNP probe coordinates

| Probe_ID | chrom: start_end |
|---|---|
| SNP_P001 | chr1: 45973869-45973988 |
| SNP_P002 | chr1: 50666456-50666575 |
| SNP_P003 | chr1: 158582587-158582706 |
| SNP_P004 | chr1: 167849355-167849474 |
| SNP_P005 | chr1: 179520447-179520566 |
| SNP_P006 | chr1: 209811827-209811946 |
| SNP_P007 | chr1: 209%8625-209%8744 |
| SNP_P008 | chr2: 44502729-44502848 |
| SNP_P009 | chr2: 169788957-169789076 |
| SNP_P010 | chr2: 170092336-170092455 |
| SNP_P011 | chr2: 179454335-179454454 |
| SNP_P012 | chr2: 179455148-179455267 |
| SNP_P013 | chr2: 215819954-215820073 |
| SNP_P014 | chr2: 2278%917-227897036 |
| SNP_P015 | chr4: 5749845-5749%4 |
| SNP_P016 | chr4: 83582005-83582124 |
| SNP_P017 | chr4: 86844776-86844895 |
| SNP_P018 | chr4: 86915789-86915908 |
| SNP_P019 | chr4: 88534176-88534295 |
| SNP_P020 | chr5: 13718%3-13719082 |
| SNP_P021 | chr5: 13829740-13829859 |
| SNP_P022 | chr5: 13844986-13845105 |
| SNP_P023 | chr5: 41000284-41000403 |
| SNP_P024 | chr5: 53751929-53752048 |
| SNP_P025 | chr5: 55155343-55155462 |
| SNP_P026 | chr5: 82834571-82834690 |
| SNP_P027 | chr5: 129521067-129521186 |
| SNP_P028 | chr5: 135392367-135392486 |
| SNP_P029 | chr5: 138456756-138456875 |
| SNP_P030 | chr5: 171849412-171849531 |
| SNP_P031 | chr6: 71546643-71546762 |
| SNP_P032 | chr6: 146755081-146755200 |
| SNP_P033 | chr6: 152464780-152464899 |
| SNP_P034 | chr6: 152466615-152466734 |
| SNP_P035 | chr6: 152675795-152675914 |
| SNP_P036 | chr7: 34009887-34010006 |
| SNP_P037 | chr7: 55214289-55214408 |
| SNP_P038 | chr7: 106799938-106800057 |
| SNP_P039 | chr8: 104337037-104337156 |
| SNP_P040 | chr9: 77415225-77415344 |
| SNP_P041 | chr9: 100190721-100190840 |
| SNP_P042 | chr9: 136304438-136304557 |
| SNP_P043 | chr10: 69926038-69926157 |
| SNP_P044 | chr10: 78944531-78944650 |
| SNP_P045 | chr10: 85971984-85972103 |
| SNP_P046 | chr10: 1045%865-1045%984 |
| SNP_P047 | chr10: 104814103-104814222 |
| SNP_P048 | chr10: 105819897-105820016 |
| SNP_P049 | chr10: 113920406-113920525 |
| SNP_P050 | chr11: 662%06-6629725 |
| SNP_P051 | chr11: 16133354-16133473 |
| SNP_P052 | chr11: 30255126-30255245 |
| SNP_P053 | chr12: 993871-993990 |
| SNP_P054 | chr12: 52200683-52200802 |
| SNP_P055 | chr13: 39433547-39433666 |
| SNP_P056 | chr14: 5076%58-50769777 |
| SNP_P057 | chr14: 64637088-64637207 |
| SNP_P058 | chr14: 74992741-74992860 |
| SNP_P059 | chr15: 34528889-34529008 |
| SNP_P060 | chr15: 89401556-89401675 |
| SNP_P061 | chr15: 89402537-89402656 |
| SNP_P062 | chr16: 68713671-68713790 |
| SNP_P063 | chr16: 68713764-68713883 |
| SNP_P064 | chr16: 68729726-68729845 |
| SNP_P065 | chr16: 70546175-70546294 |
| SNP_P066 | chr17: 10535959-10536078 |
| SNP_P067 | chr17: 10542412-10542531 |
| SNP_P068 | chr17: 42449730-42449849 |
| SNP_P069 | chr17: 71192604-71192723 |
| SNP_P070 | chr17: 71197689-71197808 |
| SNP_P071 | chr17: 71503577-715036% |
| SNP_P072 | chr18: 21413810-21413929 |
| SNP_P073 | chr18: 47455864-47455983 |
| SNP_P074 | chr19: 10267018-10267137 |
| SNP_P075 | chr19: 12989501-1298%20 |
| SNP_P076 | chr19: 13445149-13445268 |
| SNP_P077 | chr19: 16591405-16591524 |
| SNP_P078 | chr19: 33353405-33353524 |
| SNP_P079 | chr19: 38994851-38994970 |
| SNP_P080 | chr19: 55441843-55441%2 |
| SNP_P081 | chr20: 6100029-6100148 |
| SNP_P082 | chr20: 19970646-19970765 |
| SNP_P083 | chr20: 35864995-35865114 |
| SNP_P084 | chr20: 52786160-52786279 |
| SNP_P085 | chr21: 44323531-44323650 |
| SNP_P086 | chr21: 469082%-46908415 |
| SNP_P087 | chr21: 47773044-47773163 |
| SNP_P088 | chr22: 21141241-21141360 |
| SNP_P089 | chr22: 37469532-3746%51 |
| chrX_001 | chrX: 64655551-64655671 |
| chrX_002 | chrX: 112112657-112112777 |
| chrX_003 | chrX: 112112774-112112894 |
| chrX_004 | chrX: 149711007-149711127 |
| chrY_001 | chrY: 2655336-2655456 |
| chrY_002 | chrY: 7867768-7867888 |
| chrY_003 | chrY: 14102685-14102805 |
| chrY_004 | chrY: 14937651-14937771 |
| chrY_005 | chrY: 15435417-15435537 |
| chrY_006 | chrY: 15435537-15435657 |

S5: Obtain personalized combined panel sequencing data of the patient's tumor tissue sample DNA, blood cell sample DNA and plasma cfDNA, that is: construct a plasma cfDNA library, and mix the tumor tissue sample DNA library, blood cell sample DNA library and plasma cfDNA library according to the mass ratio of 2:1:(6˜12) for different sample type libraries; obtain the captured DNA library through CCP probe hybridization capture, sequence the captured DNA library, and obtain the personalized combined panel sequencing data of tumor patients. The specific steps include:

S51: Construction of plasma cfDNA prelibrary. This application uses Roche's KAPA Hyper Prep kit (KK8504) to perform end repair, A addition and adapter ligation reactions on plasma cfDNA, and uses Roche's KAPA HiFi HotStart ReadyMix (KK2602) kit for preamplification reaction. The preamplification product is purified into a new EP tube using Beckman's AMPure XP beads to obtain the plasma cfDNA prelibrary. In the example, the plasma cfDNA after end repair and A treatment is also subjected to unique molecular identifiers (UMI) connector connection processing, and the repetitive sequences are removed by the unique molecular tags, which can improve the accuracy of single-point detection of plasma free ctDNA and solve the problem that the data in the prior art cannot be accurately removed from duplicates. In the example, specifically: after the end repair plus A PCR reaction is completed, centrifuge and add 5 μL of diluted UMI connector solution, then add 45 μL of ligation mixture (5 μL ultrapure water+30 μL ligation buffer+10 μL DNA ligase), shake to mix, centrifuge and place in a PCR instrument at 20° C. for incubation for 30 min. The DNA product after the ligation reaction was then purified using Beckman's AMPure XP beads into a new EP tube for the next step of pre-amplification. In the example, the DNA library can also be subjected to Qubit concentration detection and Agilent 2100 quality inspection, and the plasma cfDNA library can be quantified using a nucleic acid concentration detector so that the plasma cfDNA library is ≥1000 ng; and the library is analyzed using a bioanalyzer, and the main peak of the plasma cfDNA library should be between 150 and 400 bp.

S52: CCP probe hybridization capture to obtain a post-capture DNA library (CCP library), and CCP probes are used to capture target region fragments to construct a post-capture DNA library. In the example, specifically: the tumor tissue sample DNA library, the blood cell sample DNA library and the plasma cfDNA library are mixed according to the mass ratio of 2:1:(6˜12) for different sample type libraries. By mixing in this ratio, the data volume of tumor tissue sample DNA, blood cell sample DNA and plasma cfDNA 1:1:(3˜6) can be obtained, and the balance between sequencing depth and cost can be taken into account at the same time. While reaching an ultra-high depth of 100,000× for plasma, the tissue can reach a depth of 10,000× to obtain a more accurate tissue mutation spectrum, and the depth of more than 10,000× for blood cells can assist plasma in eliminating the interference of clonal hematopoiesis. After mixing, the mixture is placed in a vacuum centrifugal concentrator at 60° C. for about 20 min to obtain an evaporated library, and a DNA hybridization system and a CCP hybridization probe are added to the evaporated DNA library. After shaking and mixing, centrifugation is performed and then incubated at room temperature, and hybridization is performed according to the hybridization reaction conditions of 95° C. for 30 s, 65° C. for 4 h, and 65° C. for 16 hours. The hybridized DNA library was captured by hybridization in the target region and eluted after hybridization using the commercially available kit xGen™ Hybridization and Wash Kit (1080584). The beads with the target region fragments after elution were then amplified after hybridization using the KAPA HiFi HotStart ReadyMix (KK2602) kit. Finally, the pre-amplification product was purified into a new EP tube using Beckman's AMPure XP beads, which is the DNA library after CCP probe hybridization capture (CCP library). In an example, the final capture library can also be subjected to Qubit concentration detection. In the example, the elution after the target region hybridization is performed by using an elution method with increasing volume gradient, which can obtain higher target ratio data compared with conventional equal volume elution. In an example, the elution method with increasing volume gradient comprises the following steps: after the incubation is completed, 100 μL of 65° C. preheated washing buffer I is added, mixed and placed on a magnetic stand for 1 min until the liquid is clarified, the supernatant is discarded, and the residual liquid is discarded after instant separation; 145 μL of 65° C. preheated Stringent washing buffer is added, mixed by pipetting and incubated at 65° C. for 5 min, placed on a magnetic stand for 1 min until the liquid is clarified, and the supernatant is discarded; 150 μL of 65° C. preheated Stringent washing buffer is added, mixed by pipetting and incubated at 65° C. for 5 min, placed on a magnetic stand for 1 min until the liquid is clarified, the supernatant is discarded, and the residual liquid is discarded after instant separation; 50 μL of clear washing buffer I placed at room temperature is added, the magnetic beads are gently pipetted to resuspend, the resuspended magnetic beads are transferred to a new PCR tube, and 100 Add 155 μL of room temperature washing buffer II, oscillate and centrifuge twice, place on the magnetic stand for 1 min until the liquid is clear, and discard the supernatant; remove the PCR tube from the magnetic stand, centrifuge and place on the magnetic stand, and use a 10 μL pipette to completely discard the residual liquid at the bottom of the centrifuge tube; add 160 μL of room temperature washing buffer III, oscillate and centrifuge continuously, place on the magnetic stand for 1 min until the liquid is clear, discard the supernatant, centrifuge and place on the magnetic stand to discard the residual liquid; add 20 μL of ultrapure water to the PCR tube for elution, transfer to a new PCR tube, obtain the captured library, and proceed to the next step of amplification. After hybridization is completed, off-target reads in the system or adsorbed on the tube wall need to be cleaned. Conventional operation steps all use the same volume of solution for cleaning. This application tests that the gradient volume increase method can effectively increase the cleaning of off-target reads adsorbed on the tube wall during the previous step of blowing or swirling cleaning, and ultimately presents a target ratio that is 7.18% higher than conventional operations, and achieves a higher depth and corresponding detection sensitivity.

S53: CCP library sequencing to obtain personalized combination panel sequencing data. In the example, specifically: a gene sequencer is used to sequence the captured CCP DNA library after amplification to obtain sequencing data of tumor tissue samples, blood cell samples and plasma cfDNA samples. In an example, a tumor tissue sample DNA library, a blood cell sample DNA library and a plasma cfDNA library are mixed at a mass ratio of 2:1:6 to obtain a data volume of 1:1:3 for tumor tissue sample DNA, blood cell sample DNA and plasma cfDNA. In the example, the tumor tissue sample DNA library, the blood cell sample DNA library and the plasma cfDNA library are mixed at a mass ratio of 2:1:9 to obtain a data volume of 1:1:4 for the tumor tissue sample DNA, the blood cell sample DNA and the plasma cfDNA. In an example, a tumor tissue sample DNA library, a blood cell sample DNA library and a plasma cfDNA library are mixed at a mass ratio of 2:1:12 to obtain a data volume of 1:1:6 for tumor tissue sample DNA, blood cell sample DNA and plasma cfDNA. Conventional genetic testing methods all use sequencing depths of several hundred or several thousand×for testing. As MRD tissue prior strategies are further studied in clinical practice, various research institutions expect to use fixed panel higher-depth sequencing to improve the sensitivity of MRD detection. Due to cost pressure, the 30,000× plasma sequencing depth is currently more commonly used. The present invention adopts the patient's personalized tissue panel (relatively small panel) for personalized tracking detection, so it is possible to use 100,000× ultra-high depth to perform tracking detection of MRD personalized mutation spectrum while effectively controlling costs. It can strike a balance between sequencing depth and cost.

S6: Correction of tracking mutation signals and determination of tracking mutation sequences and positions, namely: using the personalized combined panel sequencing data of tumor tissue samples and blood cell samples to correct tracking mutation signals, remove signals that are no longer determined to be somatic small mutations and fusion mutations, remove mutations of clonal hematopoietic origin, update tracking mutation signals to generate final tracking mutation signals and determine the final tracking mutation signal sequence and position, specifically including the following steps:

S61: Refer to steps S2 and S3 to process the personalized combined panel sequencing data, obtain a new tracking mutation signal, and match whether the tracking mutation signal in S3 is in the new tracking mutation signal, delete the mutation signal that does not exist in the new tracking mutation signal, and generate a final tracking mutation signal. As mentioned above, the tissues and blood cells in the WDC combination sequencing have a sequencing depth of only 200×, while the tissues and blood cells in the CCP combination sequencing data have a sequencing depth of more than 10,000×. The high depth can locate the site frequency in the tissue more accurately, and the clonal hematopoiesis detected in the high-depth blood cells can be excluded at the same time. That is, the tissue mutation spectrum is finely screened through the personalized combination panel sequencing data, making the sample detection more accurate.

S64: Determine the final tracking mutation sequence and position according to the following method: obtain the extended mutation sequence, that is, first, based on the reference genome and the final tracking mutation signal, for each tracking mutation sequence, concatenate the three sequences of the reference genome sequence from its starting position to 3 bp upstream of the genome, the tracking mutation sequence and its ending position to 3 bp downstream of the genome as the candidate sequence; if the candidate sequence can only be uniquely matched within the range of 200 bp upstream and downstream of the candidate sequence, then retain the candidate sequence as the tracking mutation sequence, and at the same time define the genome starting position of the concatenated sequence as the genome starting position of the tracking mutation sequence, and the genome ending position of the concatenated sequence as the genome ending position of the tracking mutation sequence; if the retention standard is not met, increase the length by 1 bp, that is, start to re-extend the upstream and downstream sequences from 4 bp and repeat the operation until the retention standard is met or the length of the concatenated sequence exceeds 35 bp. The only sequence containing the tracking mutation near the tracking mutation signal is determined, effectively avoiding the possibility of matching other nearby positions. Upstream and downstream extensions increase the possibility of the existence of such unique fragments, while longer fragments can be matched and positioned more accurately. On the other hand, directly using the mutated sequence for upstream and downstream extension can more directly determine whether each sequencing sequence (read) or single-stranded consensus sequence (SSCS) supports the tracking mutation signal. The traditional method of comparing with the reference gene sequence cannot accurately compare and locate, especially when long fragments are inserted or deleted. For Indels, especially long fragment insertions and deletions, this application can effectively improve the matching and positioning accuracy.

S7: Obtain the final tracking mutation signal detection result of plasma cfDNA, that is, extract the reads pair of the plasma sample covering the final tracking mutation signal position, extract the molecular tag sequences at both ends, the starting position on the genome, the length and direction of the inserted fragment and other information, determine the single-stranded consensus sequence and the double-stranded consensus sequence, and determine the tracking mutation signal detection result, which specifically includes the following steps:

S71: Remove the adapter, extract the UMI sequence and align it, and extract the reads of the plasma sample covering the final tracking mutation signal position. In the example, the adapters are removed and the UMI sequences are extracted, and fastp (0.23.2) is called to treat each pair of FASTQ files as paired reads to remove the adapters and UMI sequences, and the parameters “--trim_poly_g --poly_g_min_len 10 --cut_right --cut_window_size 4 --cut_mean_quality 20 --overlap_len_require 30 --overlap_diff_limit 5 --overlap_diff_percent_limit 20 --length_required 51 --adapter_fasta adapters/TruSeq3-PE.f” are used to generate FASTQ files after the adapters are removed and the UMI sequences are extracted, and the extracted UMI sequences exist in the IDs of the corresponding reads. Align and extract UMI sequences, call the commercial software Sentieon-202112.05 to use the FASTQ files after removing the adapters as paired reads, use the umi extract module to extract UMI sequences, use the bwa men module to align to the hg19 human reference genome sequence, and use the util sort module to sort the alignment results to generate the initial Bam file. In an example, if it is double-end sequencing, a pair of reads with the same read ID number is marked as 1 fragment, and information of the fragment including the UMI sequences at both ends, the starting position on the genome, the length and direction of the inserted fragment is extracted.

S72: Determine the single-stranded consensus sequence (SSCS), and group the fragments with matching fragment information, wherein the matching fragment information refers to the UMI sequence, starting position or insert fragment difference within the error range of 1 bp, and having almost identical fragment information, starting from the base position on the fragment corresponding to the genome starting position of the final tracking mutation signal sequence, to the base position on the fragment corresponding to the genome ending position of the tracking mutation sequence, and comparing the number of each base type at each position base by base, the base types include A, T, C, G,; determine the SSCS, if Bₘₐₓ/B_{second}>2 is satisfied, the base type of the SSCS at this position is the base type with the largest number, and the base type of the negative consensus sequence at this position is marked as N, where Bₘₐₓ represents the number of the base type with the largest number, and B_{second} represents the number of the second largest base type.

S73: Determine the type of supported tracking mutations: For each tracking mutation, define the SSCS that completely matches the tracking mutation signal sequence as a simplex, and define two simplexes with paired UMI sequences as a duplex.

S74, filtering and determining tracking mutations: Filter tracking mutations according to the following rules: If the minimum value of the distance between the edge of the tracking mutation on the simplex and the edge of the fragment is less than 5, or the number of bases on the simplex that are different from the reference genome sequence is greater than 5, then the simplex is defined as a low-quality simplex. The proportion of low-quality simplex for each tracking mutation is counted. If it is greater than 0.5, the mutation is considered to be a low-confidence mutation and is removed in subsequent analysis. The number of simplexes and duplexes of each tracking mutation after filtering is counted. If the number of simplexes is greater than 0 and the number of duplexes is greater than 1, the mutation is reported as a positive mutation.

S8: Obtain the MRD test results, and combine the test results of all tracking mutation signals to obtain the MRD test results of the patient to be tested. That is, if there are still positive mutations greater than the preset threshold number after the above strict filtering, the patient's MRD status is defined as positive, otherwise it is negative. In an example, the threshold value=1 is preset.

### Example 2

This example provides the sequencing depth of each region of WDC probe hybridization capture and sequencing by preparing WDC probes by mixing whole exome sequencing probes (WES probes) with targeted drug gene panels in different ratios. For other steps, refer to Example 1.

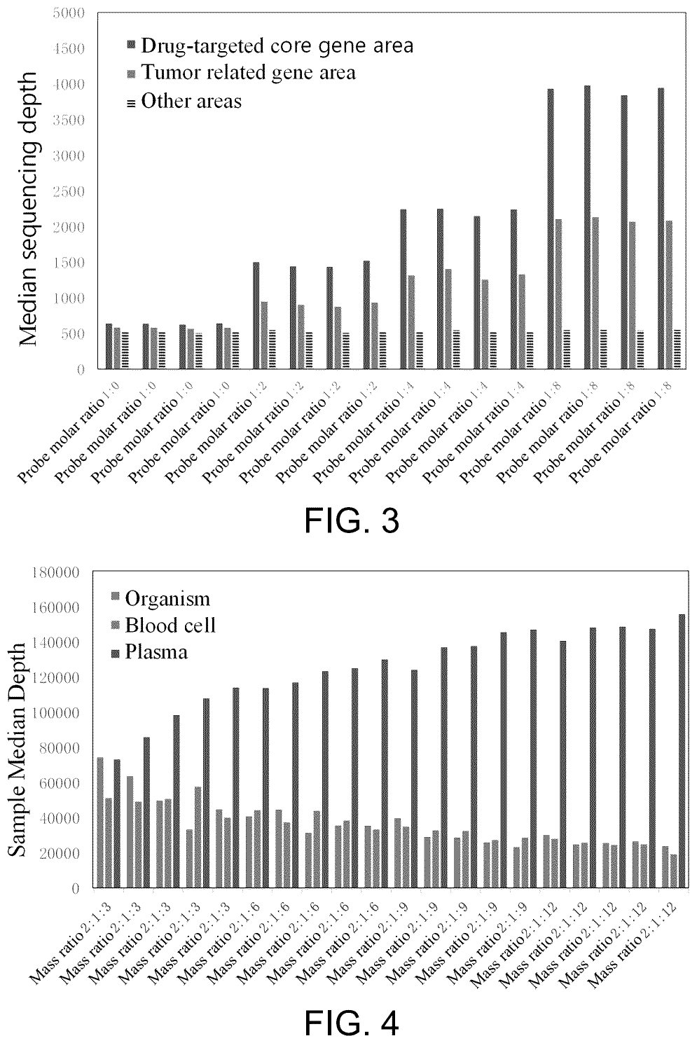

The results are shown in FIG. 3, and WDC probes can achieve differentiation in sequencing depth. Compared with sequencing data captured only by WES probe hybridization, the WDC combination panel can achieve an effective depth ratio of 1:(1.5˜3):(2˜6) for WES other regions:tumor-related gene regions:targeted drug core gene regions, which can reduce the detection limit of targeted drug core genes and tumor-related genes, thereby improving the sensitivity of tissue detection.

### Example 3

This example provides sequencing depths after CCP probe hybridization capture after mixing tumor tissue sample DNA libraries, blood cell sample DNA libraries and plasma cfDNA libraries in different proportions. For other steps, refer to Example 1.

The results are shown in FIG. 4. A ratio lower than 2:1:(6-12) requires an increase in the amount of sequencing data and sequencing costs to achieve high-depth equivalence for plasma, while a ratio higher than this requires a higher amount of data to achieve deep equivalence for tissues and blood cells. Since in the co-capture system of tissue, blood cells and plasma, the degree of damage to tissue fragments is greater than that of blood cells, the input amount needs to be more than that of blood cells. At the same time, the input amount of tissue and blood cells (30 ng-300 ng) is higher than that of plasma (10 ng-50 ng), so plasma requires a higher data volume to achieve a higher sequencing depth. The present invention takes into account cost factors and ultra-high depth requirements, and ultimately determines that when the tumor tissue sample DNA library, blood cell sample DNA library and plasma cfDNA library are subjected to CCP probe hybridization capture at a mass ratio of 2:1:(6-12), the sequencing cost can be controlled while the median depth of plasma samples can reach 100,000× data depth, and the median depth of tissue and blood cells can reach 10,000× data depth.

### Example 4

This example provides the comparison results of elution after hybridization of the target region in the CCP probe hybridization capture process using a volume gradient elution method and a conventional equal volume elution method. For other steps, refer to Example 1.

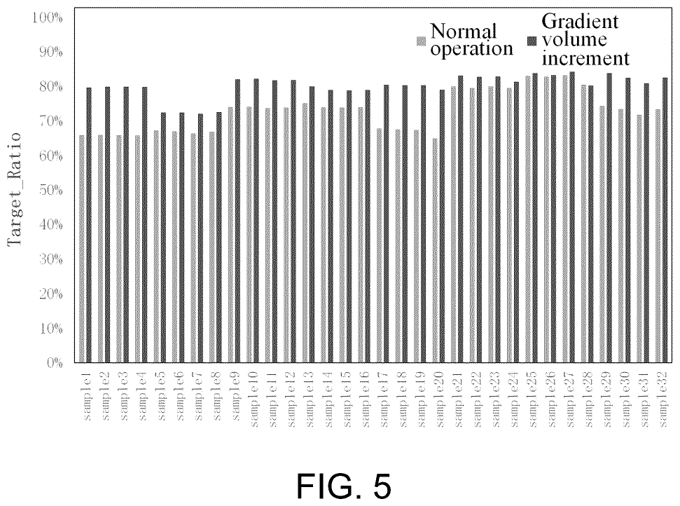

The results are shown in FIG. 5. Compared with the conventional operation steps, the same volume of cleaning solution is used for cleaning. The method of increasing the gradient volume in this example can effectively increase the cleaning of off-target reads adsorbed on the tube wall during the blowing or swirling cleaning process in the previous step, and finally presents a target ratio that is 7.18% higher than that of conventional operation, and achieves a higher depth and corresponding detection sensitivity.

### Example 5

This example provides a detection device for micro residual lesions, including:

A data input module, used to input the WDC sequencing data of the patient's tumor tissue sample and preoperative blood cell sample in Example 1, and input the personalized combined panel sequencing data of the patient's tumor tissue sample, blood cell sample and plasma;

A data processing module, used to complete the acquisition of patient genome mutation signals, screening of tracking mutation signals, tracking mutation signal correction, determination of tracking mutation sequences and positions, and acquisition of tracking mutation signal detection results of plasma cfDNA as described in Example 1 according to input data;

The result output module is used to output the MRD detection results of the tumor patient described in Example 1.

### Comparative Example 1

The patent number of this comparative example is CN109477138A, while its invention name is lung cancer detection method. It detects preoperative blood plasma samples of 51 stage I lung cancer patients with tumors. Refer to CN109477138A for more details of this method.

Result analysis:

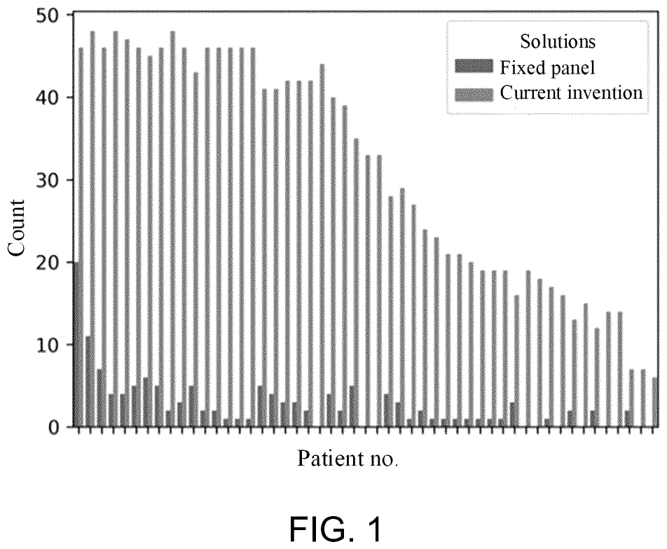

The number of traceable mutations detected in Example 1 and Comparative Example 1 is shown in FIG. 1. In the current application, 1,794 mutations can be traced in 51 samples, with an average of 35 mutations per sample (median 39), while 168 mutations can be traced in 51samples of Comparative Example 1, with an average of 3 mutations per sample (median 2). This shows that the current application has a greater number of traceable mutations.

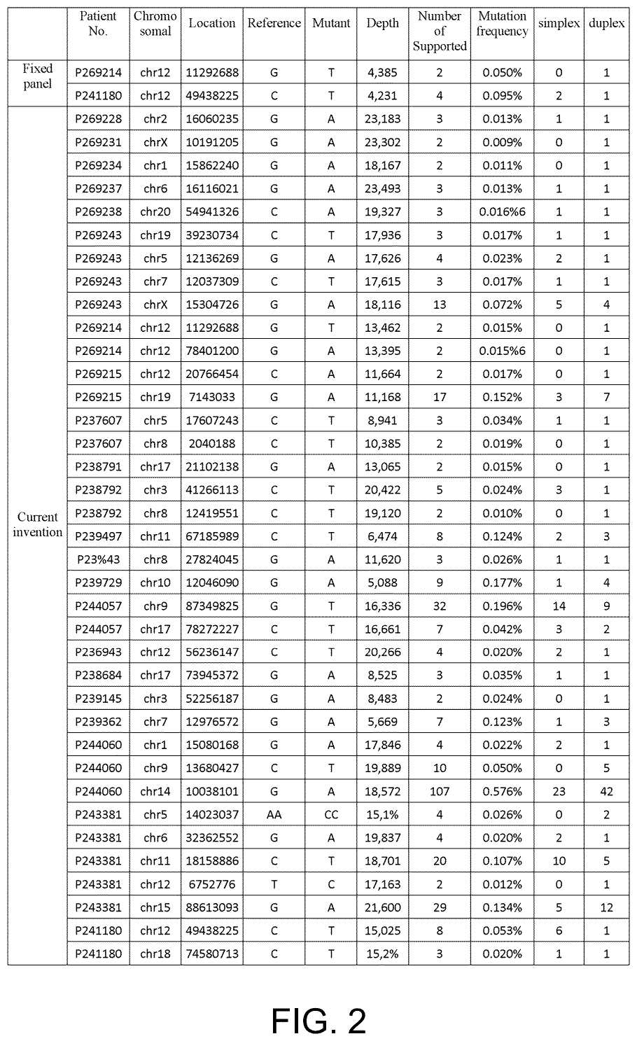

The positive mutations detected in Example 1 and Comparative Example 1 are shown in FIG. 2. Among the 51 samples in the current application, 37 positive mutations were detected in 22 samples, while only 2 positive mutations were detected in 2 samples in Comparative Example 1. This shows that the current application scheme detects more positive mutations.

The positive detection rate of Example 1 and Comparative Example 1 is calculated by the following formula:

$$Positive Detection Rate = \frac{Number of patients tested positive}{Number of postitive patients}$$

The positive detection rate of this application is 22/51=43.13%, while the positive detection rate of comparative example 1 is 2/51=3.9%, which is a very significant improvement. At the same time, compared with the results of all other institutions that can be publicly queried, the average positive detection rate is mostly below 10%, and this application has a significant effect.

What is claimed is:

1. A detection device for MRD lesions, comprising:
data input module, which is used to obtain WDC sequencing data of a patient's tumor tissue sample and preoperative blood cell sample, and to input personalized combined panel sequencing data of a patient's tumor tissue sample, blood cell sample, and plasma;
data processing module, which is used to obtain genome mutation signals, screen tracking mutation signals, correct tracking mutation signals, determine tracking mutation sequences and positions, and obtain tracking mutation signal detection results of plasma cfDNA according to input data;
result output module, which is used to output MRD detection results;
method for obtaining the WDC sequencing data comprises:
S1, obtain WDC sequencing data of tumor tissue DNA and blood cell DNA of patients, and construct tumor tissue DNA library and blood cell DNA library respectively; mix two libraries with equal mass ratio, and use WDC probe for hybridization capture to obtain captured DNA library, wherein WDC probe is a mixed probe formed by mixing whole exome sequencing probe with targeted drug gene panel in a molar ratio of 1:(2-8), and genes in the targeted drug gene panel include one or more genes from AKT1, ALK, AR, ARAF, BRAF, BRCA1, BRCA2, CDK4, CTNNB1, DDR2, EGFR, ERBB2, ERBB3, ERRFI1, ESR1, FBXW7, FGFR1, FGFR2, FGFR3, FLT1, GNA11, GNAQ, HRAS, IDH1, IDH2, KIT, KRAS, MAP2K1, MAPK1, MET, MTOR, NF1, NF2, NOTCH1, NRAS, NTRK1, NTRK2, NTRK3, PDGFRA, PIK3CA, PTEN, RAC1, RB1, RET, RICTOR, ROS1, SMAD4, TERT, TP53, TSC1, VEGFA, AKT2, AKT3, APC, ATM, ATR, ATRX, CDK6, CDKN2A, CHEK2, FLT3, FLT4, JAK1, JAK2, KDR, KEAP1, MDM2, MYC, PALB2, VHL, ABL1, BTK, SMO, ETV6, EWSR1, NTRK, HER2 and BRCA; sequencing the captured DNA library to obtain WDC sequencing data of tumor patients, wherein the WDC sequencing is differentiated in depth for WES+targeted drug gene panel sequencing,
obtaining of the genome mutation signals comprises:
S2, obtaining a patient's genome mutation signal by: pre-processing the WDC sequencing data obtained in S1, aligning it with a hg19 human reference genome, removing duplicates, re-aligning, and correcting its quality value to obtain a DNA mutation signal of the tumor tissue sample and the DNA mutation signal of the blood cell sample, comparing and retaining the DNA mutation signal that only exists in the tumor tissue sample as the genome mutation signal, the DNA mutation signal includes one or more of somatic cell variation, insertion and deletion, fusion, or other types of mutation;
screening of tracking mutation signals comprises:
S3, screening the tracking mutation signals by: sorting the genome mutation signals in S2 according to function and credibility, screening a preset number of genome mutation signals with a highest ranking as tracking mutation signals, and sorting rules are as follows: firstly, driver mutations with important functions are given the highest ranking priority; secondly, sort them by mutation frequency and primary clone-subclone, for mutations with a mutation frequency greater than 5%, sort them from large to small according to mutation frequency; for mutations with a mutation frequency between 1% and 5%, sort them by primary clone>subclone first, and then by mutation frequency second;
method for personalized combined panel sequencing data acquisition comprises:
S4, design a tracking mutation signal sequence probe based on the tracking mutation signal, and mix it with a fixed mutation signal sequence probe and SNP probe to prepare a personalized combination panel, where the fixed mutation signal sequence probe is used to detect tumor evolution or second primary, and the SNP probe is used to identify a source of the sample and evaluate a degree of sample contamination;
S5, obtaining personalized combined panel sequencing data of the patient's tumor tissue sample DNA, blood cell sample DNA and plasma cfDNA: constructing a plasma cfDNA library containing UMI connectors, and mixing different sample type libraries of tumor tissue sample DNA library, blood cell sample DNA library and plasma cfDNA library at a mass ratio of 2:1:(6-12); obtaining a captured DNA library through CCP probe hybridization capture, sequencing the captured DNA library to obtain personalized combined panel sequencing data of tumor patients;
correcting tracking mutation signals and determining tracking mutation sequences and positions comprises:
S6, correcting tracking mutation signals and determining tracking mutation sequences and positions by: using personalized combined panel sequencing data of tumor tissue samples and blood cell samples to correct tracking mutation signals, remove signals that are no longer determined to be somatic small mutations and fusion mutations, remove mutations of clonal hematopoietic origin, update tracking mutation signals to generate final tracking mutation signals and determine sequence and position of the final tracking mutation signals;
method for obtaining tracking mutation signal detection results of plasma cfDNA comprises:
S7, obtaining tracking mutation signal detection results of plasma cfDNA by: extracting the reads pairs of a plasma sample covering the final tracking mutation signal position, extract molecular tag sequences at both ends, a starting position on the genome, a length and direction of an inserted fragment, determine a single-stranded consensus sequence and a double-stranded consensus sequence, filter and determine the tracking mutation signal detection results in combination with a UMI sequence;
obtaining of the MRD detection result includes:
S8, combining the detection results of all tracking mutation signals to obtain the MRD detection results of the tumor patient: counting a number of positive mutations of the tracking mutation signal in S7, and comparing it with a preset threshold, if greater than the preset threshold, MRD status of the tumor patient is positive, otherwise MRD status of the tumor patient is negative.

2. The detection device for MRD lesions according to claim 1, wherein the genome mutation signal obtained in S2 also includes filtering, and filtering rules are as follows: a population mutation frequency of three databases of gnomAD, ExAC, and 1000 g is less than 2%; a sequencing depth is greater than 40; a mutation frequency is greater than 1%; it is not in the platform blacklist range which contains repeated mutations with low quality collected among different batches of samples with large amount; it supports reads>2, coverage depth>100, there is no significant difference in positive and negative chain support, there is no simple repeat sequence in and around it, and a tumor tissue mutation frequency/blood cell mutation frequency>5.

3. The detection device for MRD lesions according to claim 1, wherein classification between primary clone and subclone in S3 is based on the genome mutation signal and CNV detection results in S2, the number of supporting mutation reads and sequencing depth of each somatic cell mutation is used to estimate a tumor purity and group the somatic cell mutations into different clone populations, and cell proportion of each clone population is counted, the clone population with a highest proportion is defined as the main clone, and other categories are defined as subclones; the CNV detection results are comparation between tumor tissue samples and blood cell samples to obtain estimated values of tumor purity of tumor tissue samples and tumor cell allele copy number.

4. The detection device for MRD lesions according to claim 3, wherein design rules of the tracking mutation signal sequence probe in S4 are as follows: if it is a SNV/Indel type mutation, according to the reference genome and the tracking mutation list, the reference genome sequence 60 bp upstream of the genome at the starting position of each tracking mutation signal, the tracking mutation signal sequence and the reference genome sequence 60 bp downstream of the genome at the ending position of the tracking mutation signal are concatenated in series as candidate tracking mutation signal probe sequences; if it is a Fusion type mutation, according to the reference genome and the direction of the fusion mutation, the sequence 60 bp upstream of a breakpoint 1 of the upstream gene gene1 of the fusion mutation and the sequence 60 bp downstream of the breakpoint 2 of the downstream gene gene2 of the fusion mutation along a transcription direction are concatenated in series as a candidate tracking mutation signal probe sequence; the fixed mutation signals in the fixed mutation signal sequence probe include targeted evidence gene sites and chemotherapy resistance evidence gene sites from NCCN guidelines, expert consensus, and public databases, FDA/NMPA drug labels, clinical trials and conference abstract evidence gene sites, and one or more of the sets formed by screening out first-level evidence gene sites and second-level evidence gene sites in multiple cancer types; the SNP probe site includes one or more of the sets of SNPs sites with higher heterozygosity from the dbSNP database covered by the whole exome in WDC.

5. The detection device for MRD lesions according to claim 4, wherein the design of the tracking mutation signal sequence probe in S4 also includes filtering, and filtering rules are as follows: remove candidate probe sequences with more than 20 “better matching positions” in the entire reference genome, wherein the “better matching positions” refer to positions with a matching length greater than 30 bp and a matching expectation value less than 0.000001; remove candidate probe sequences containing repetitive sequence SSRs; remove abnormal candidate sequences with GC<10% or GC>80%.

6. The detection device for MRD lesions according to claim 5, wherein after the hybridization capture in S5 is completed, elution is performed in a volume gradient increasing manner to obtain a hybridization captured DNA library.

7. The detection device for MRD lesions according to claim 6, wherein the tracking mutation signal correction in S6 comprises: referring to S2 and S3 to process the personalized combined panel sequencing data, obtaining a new tracking mutation signal, and matching whether the tracking mutation signal in S3 is in the new tracking mutation signal, deleting the mutation signal that does not exist in the new tracking mutation signal, and generating a final tracking mutation signal;
determining the final tracking mutation sequence and position includes: obtaining an extended mutant sequence, and according to the reference genome and the final tracking mutation signal, for each tracking mutation sequence, concatenating the reference genome sequence from its starting position to the upstream length abp of the genome, the tracking mutation sequence and its ending position to the reference genome sequence from the downstream abp of the genome in series as candidate sequences; if the candidate sequence can only be uniquely matched within a range of bbp including the upstream and downstream of the candidate sequence, then the candidate sequence is retained as the tracking mutation sequence, and the genome starting position of the concatenated sequence is defined as the genome starting position of the tracking mutation sequence, and a genome ending position of the concatenated sequence is defined as the genome ending position of the tracking mutation sequence; if a retention standard is not met, then the length is increased by 1 bp, that is, (a+1) bp is used to re-extend the upstream and downstream sequences and then the operation is repeated until the retention standard is met or the length of the concatenated sequence exceeds cbp, where a is 3˜4, b is 100˜200, and c is 30˜35.

8. The detection device for MRD lesions according to claim 7, wherein the determining of the single-stranded consensus sequence in S7 comprises: marking a pair of reads with the same read ID number as a fragment; grouping the fragments with matching fragment information, wherein the matching fragment information refers to the UMI sequence, the starting position or the difference of the inserted fragment within the error range of d bp, and having almost completely identical fragment information; starting from a base position on the fragment corresponding to the genome starting position of the final tracking mutation signal sequence, to the base position on the fragment corresponding to the genome ending position of the tracking mutation sequence, comparing the number of each base type at each position base by base, the base types including A, T, C, and G; determining SSCS, if Bₘₐₓ/B_{second}>f is satisfied, the base type of the consensus sequence at this position is the base type with a largest number, and the base type of a negative consensus sequence at this position is marked as N, wherein Bₘₐₓ represents a number of the base type with the largest number, and B_{second} represents a number of the base type with the second largest number.

9. The detection device for MRD lesions according to claim 8, wherein the filtering and determining the tracking mutation signal detection result in combination with the UMI sequence in S7 comprises: for each tracking mutation, defining a single-stranded consensus sequence that completely matches the tracking mutation sequence as a simplex, and defining two simplexes with paired molecular tag sequences as a duplex; filtering and determining the tracking mutation according to following rules: if a smaller value of the tracking mutation edge distance to the fragment edge distance on the simplex is less than a preset threshold j, or the number of bases on the simplex that are different from the reference genome sequence is greater than a preset threshold n, then the simplex is defined as a low-quality simplex; counting the proportion of low-quality simplexes of each tracking mutation, if it is greater than a preset threshold r, the mutation is considered to be a low-confidence mutation and is removed in subsequent analysis; counting the number of simplexes and the number of duplexes of each tracking mutation after filtering, if the number of simplexes is greater than a preset threshold s and the number of duplexes is greater than a preset threshold h, then the mutation is reported as a positive mutation.

10. An electronic device, wherein it comprises: one or more processors; a storage device on which one or more programs are stored, and when the one or more programs are executed by the one or more processors, the one or more processors implement S1 to S8 in the detection device for detecting micro residual lesions according to claim 1.

11. A computer storage medium, wherein a computer program is stored thereon, wherein when the computer program is executed by a processor, S1 to S8 in the detection device for MRD lesions according to claim 1 are implemented.


SEQUENCE LISTING (37 sequences)

SEQ ID NO: 1 - DNA, 120 bp, synthetic construct
  source 1..120: mol_type = other DNA; note = synthetic DNA probe; oligonucleotide; designed for Human Genome target region capture
aagaccttgagaccttagccctaaaggcatacacctcatagcttctcacctcagagccttggtgatatcttctggcttgaagttattagatccttctagaggcttctgtaggtacccata

SEQ ID NO: 2 - DNA, 120 bp, synthetic construct
  source 1..120: mol_type = other DNA; note = synthetic DNA probe; oligonucleotide; designed for Human Genome target region capture
ctgctgtgtctgatcggggagagctttgaggaacacagcagagaggtatgtggggccgttgtcaacatccgcaccaagggggacaagatcgctgtgtggacgagggaggcggaaaaccag

SEQ ID NO: 3 - DNA, 120 bp, synthetic construct
  source 1..120: mol_type = other DNA; note = synthetic DNA probe; oligonucleotide; designed for Human Genome target region capture
aagctctgatgccatttcaccactccttttgtctcctacgaaaagttgtcccttctgcttcgggtcgggttcttgcttcccgaaacaccaagacgtcggtggtggtgcagtgggaccgac

SEQ ID NO: 4 - DNA, 120 bp, synthetic construct
  source 1..120: mol_type = other DNA; note = synthetic DNA probe; oligonucleotide; designed for Human Genome target region capture
cgcgttctgactgattccatacagagaatacagcagacataaactccttaagacagcttaaatggctttatcttgaattttgaggagtttttctgaaaagagcttaactaccacatagtg

SEQ ID NO: 5 - DNA, 120 bp, synthetic construct
  source 1..120: mol_type = other DNA; note = synthetic DNA probe; oligonucleotide; designed for Human Genome target region capture
tcaaactggtcttgggcattctccccattgtagatctcatgcacaatgtagcagtctgtagccgacaagctcacccttttgatggagggggcaggaaagggagagagagagagagacagg

SEQ ID NO: 6 - DNA, 120 bp, synthetic construct
  source 1..120: mol_type = other DNA; note = synthetic DNA probe; oligonucleotide; designed for Human Genome target region capture
cagaaaagcggctgttagtcactggcagcaacagtcttacctggactctggaatccattttggtgccactaccacagctccttctctgagtggtaaaggcaatcctgaggaagaggatgt

SEQ ID NO: 7 - DNA, 120 bp, synthetic construct
  source 1..120: mol_type = other DNA; note = synthetic DNA probe; oligonucleotide; designed for Human Genome target region capture
gctgggcctcagctgagaagccctacctgaaggaaaaatccagcgccactgtgtacttctagaccgtcaagcacaacaacatcagagacctcgtccgccgctgcatcacccggactagcc

SEQ ID NO: 8 - DNA, 120 bp, synthetic construct
  source 1..120: mol_type = other DNA; note = synthetic DNA probe; oligonucleotide; designed for Human Genome target region capture
ttggcgctaaggttgagctctcgcagctccttggccttggaaaagaagccgggggccacaaagctgatgctgttgcagctgacatccagcctccggagccgggtgccagcaggcaggctg

SEQ ID NO: 9 - DNA, 120 bp, synthetic construct
  source 1..120: mol_type = other DNA; note = synthetic DNA probe; oligonucleotide; designed for Human Genome target region capture
tttatgtatgtgtatatgttcttttttttctccaagatccatgcatacaaccttgaaacaaatgcctgggaggaaattgcaacaaaaccccatgaaaaaataggtaaatttaaagtattg

SEQ ID NO: 10 - DNA, 120 bp, synthetic construct
  source 1..120: mol_type = other DNA; note = synthetic DNA probe; oligonucleotide; designed for Human Genome target region capture
cgttctgggctcccccagcacctttctgcctgtgctgctggagggtggggtccagagcctgggtgagtgtatgctcaagtttccccatccccttctacagaaagggcagccctgccctgg

SEQ ID NO: 11 - DNA, 120 bp, synthetic construct
  source 1..120: mol_type = other DNA; note = synthetic DNA probe; oligonucleotide; designed for Human Genome target region capture
gacagcatgcttcagggccgacagggaccccagctgggtacagcagatgcttgcccgccatttgtgacatggacctggacaataagggtaagaggaggaaggaaagaagacacttaaaaa

SEQ ID NO: 12 - DNA, 120 bp, synthetic construct
  source 1..120: mol_type = other DNA; note = synthetic DNA probe; oligonucleotide; designed for Human Genome target region capture
acatgttccgtaaacagctttataaggtcatcttttaagtctctgttaagcttggtttcaatgtaaaacttattctgaaaaataaaataaaatctttttttgtgtattaattggggaaat

SEQ ID NO: 13 - DNA, 120 bp, synthetic construct
  source 1..120: mol_type = other DNA; note = synthetic DNA probe; oligonucleotide; designed for Human Genome target region capture
accccatcccaggacctcaccaggccgatatggtttacgcttgcgttttttgctttccttggtctcctctttgccaggctccacatcagggctgacggggccctccagtttaatttcgca

SEQ ID NO: 14 - DNA, 120 bp, synthetic construct
  source 1..120: mol_type = other DNA; note = synthetic DNA probe; oligonucleotide; designed for Human Genome target region capture
ctgagtgtggggatgagtttactctgcagagtcagctggccgtgcacatggaggagcactgccaggagctggctggaacccggcagcatgcctgcaaggcctgcaagaaagagttcgaga

SEQ ID NO: 15 - DNA, 120 bp, synthetic construct
  source 1..120: mol_type = other DNA; note = synthetic DNA probe; oligonucleotide; designed for Human Genome target region capture
cggtggggagttggtcgtactcgcagcagaggaggcagagggtgtgctctggcgagggtccgcagaagaccgacctcatggccttcagcccgggcctttctccttgtgctggatctacag

SEQ ID NO: 16 - DNA, 120 bp, synthetic construct
  source 1..120: mol_type = other DNA; note = synthetic DNA probe; oligonucleotide; designed for Human Genome target region capture
atttgctgaactcacagagggaagctggcttggtgaaaaggccagggatctcaggccccaaacagcccagaaatacaaggtgcctcctctctggcacttggaaagtcagaactcacaggt

SEQ ID NO: 17 - DNA, 120 bp, synthetic construct
  source 1..120: mol_type = other DNA; note = synthetic DNA probe; oligonucleotide; designed for Human Genome target region capture
gaccctgagcttcacggggctgacgtgcatcgtttcccttgtcgcgctgacaggaaacgtggttgtgctctggctcctgggctgccgcatgcgcaggaacgctgtctccatctacatcct

SEQ ID NO: 18 - DNA, 120 bp, synthetic construct
  source 1..120: mol_type = other DNA; note = synthetic DNA probe; oligonucleotide; designed for Human Genome target region capture
acctgaaggaacataaggaatcaccaacctgagagagaaaaagttgcgattttctcctcacccaaaaaggggatgctgatggaacaagtgacgtccacagcggagatgtttgtgaccctt

SEQ ID NO: 19 - DNA, 120 bp, synthetic construct
  source 1..120: mol_type = other DNA; note = synthetic DNA probe; oligonucleotide; designed for Human Genome target region capture
accaaggatctcctcctccatgtacttccaggccttggctgtgggggttatgatgcaggcattctccacgatcgaatagcacagcaccagcaaggcctctgtgtggggcagctgcaggag

SEQ ID NO: 20 - DNA, 120 bp, synthetic construct
  source 1..120: mol_type = other DNA; note = synthetic DNA probe; oligonucleotide; designed for Human Genome target region capture
ggacgtacacctggctgggcgccctgcctgtcctgcactgctgtatggagctggccccgtggcacaaggatgcctggagacagcctgaggacacctgggccgctctggagggactctcct

SEQ ID NO: 21 - DNA, 120 bp, synthetic construct
  source 1..120: mol_type = other DNA; note = synthetic DNA probe; oligonucleotide; designed for Human Genome target region capture
taatacagccctttaaggagaaaattaagtcaaaaatttgacccacctttcagagggtgtgtcagtcttagatctttctgctgtctaagctcttttcccccttctccttttcatttggaa

SEQ ID NO: 22 - DNA, 120 bp, synthetic construct
  source 1..120: mol_type = other DNA; note = synthetic DNA probe; oligonucleotide; designed for Human Genome target region capture
tcttgacctgtgggcatgacaagcatgccactctctgggacgctgtgggtcaccgtcccatctgggacaaaataatagaggtaaacatgcacattacatttccatttttcttacagaaat

SEQ ID NO: 23 - DNA, 120 bp, synthetic construct
  source 1..120: mol_type = other DNA; note = synthetic DNA probe; oligonucleotide; designed for Human Genome target region capture
atgttgtgtcgggagatgaactctgttggttgacaaacattactcatgtctgtacagttaggagaactagttacctgagagtgaagagataaaaatgaggtaaaatgataccattattta

SEQ ID NO: 24 - DNA, 120 bp, synthetic construct
  source 1..120: mol_type = other DNA; note = synthetic DNA probe; oligonucleotide; designed for Human Genome target region capture
gtgactgctcacctttccaaagtctcgcacatcgaagaggtcaaaggggtcaaacagcttgctgttccttaatccaaatttatcgtggcagactttcaggaaggtgcgtatgttcttcaa

SEQ ID NO: 25 - DNA, 120 bp, synthetic construct
  source 1..120: mol_type = other DNA; note = synthetic DNA probe; oligonucleotide; designed for Human Genome target region capture
tgttgactgcgatattgacgttcccaaaaccatccagatggtgcggtctcagaggtcagtgatggtccagacagaagcacagtaccgatttatctatatggcggtccagcattatattga

SEQ ID NO: 26 - DNA, 120 bp, synthetic construct
  source 1..120: mol_type = other DNA; note = synthetic DNA probe; oligonucleotide; designed for Human Genome target region capture
gcacaggaaatggtgctgtccaactccctcaacagcagcaacatagccacccgaataccacgacagtggcaccattcatttacaggtaaggtggcctctgtttatccacagttgtaaata

SEQ ID NO: 27 - DNA, 120 bp, synthetic construct
  source 1..120: mol_type = other DNA; note = synthetic DNA probe; oligonucleotide; designed for Human Genome target region capture
caaaaggcctgtgctcctccggactcgtgggcacgctggtcgaggaagtgttggggaaaactgccaccgtgggcacggccaccgtcacattcccaacatcgccaagggacctgcgtttcc

SEQ ID NO: 28 - DNA, 120 bp, synthetic construct
  source 1..120: mol_type = other DNA; note = synthetic DNA probe; oligonucleotide; designed for Human Genome target region capture
actgtggacatcgccgtcatgggcgaggcccacggcctcattaccgacctcctggcagaaccttctcttccaccaaacgtgtgcacatccttgagagccgtgagcaacttgctcagcaca

SEQ ID NO: 29 - DNA, 120 bp, synthetic construct
  source 1..120: mol_type = other DNA; note = synthetic DNA probe; oligonucleotide; designed for Human Genome target region capture
catagtcaagttcctagatcttcatcaatggtacttggatcatttggaacagacttaataagagagaggagagatttggagagaagaacagattcctctattagtaatcttatggattat

SEQ ID NO: 30 - DNA, 120 bp, synthetic construct
  source 1..120: mol_type = other DNA; note = synthetic DNA probe; oligonucleotide; designed for Human Genome target region capture
ttcaagcctggtccatggggtagggtcggcttcccatctataagcccctttagatttccaaaagaggcagcatctttattctgtgaaatgtttgggggcaaacccaggaacatggtactt

SEQ ID NO: 31 - DNA, 120 bp, synthetic construct
  source 1..120: mol_type = other DNA; note = synthetic DNA probe; oligonucleotide; designed for Human Genome target region capture
cagcatgtctcctgcctcataggccaataaaaattcattataacgttgcaatagacgacatctgcgttcttctgcccgatccaagagggagcggtatctggatggagaattgggaaaagt

SEQ ID NO: 32 - DNA, 120 bp, synthetic construct
  source 1..120: mol_type = other DNA; note = synthetic DNA probe; oligonucleotide; designed for Human Genome target region capture
gaatctcgaaccgcatgttcaggaaatagaagtgtctaggctgttcttggagcccacacaaaaagatattgccttgctaaagctaagcaggtactcgttcacctgtggtcttcaccccac

SEQ ID NO: 33 - DNA, 120 bp, synthetic construct
  source 1..120: mol_type = other DNA; note = synthetic DNA probe; oligonucleotide; designed for Human Genome target region capture
cgctagtctctgtgcccttgattgtcagatattttcgaaaagtgggattttttaaacctacagctgcaaaaccttaatgaactcttcagtcgtacacactgaaaacctatttcttctaaa

SEQ ID NO: 34 - DNA, 120 bp, synthetic construct
  source 1..120: mol_type = other DNA; note = synthetic DNA probe; oligonucleotide; designed for Human Genome target region capture
ttgctctacctgttccggtgattgtatccaacttcagtcgcatctaccaccagaatcaatgagcagacaaacgaagggcacaaaaggtgcgtattcaactccgtgcaaccatggtttagc

SEQ ID NO: 35 - DNA, 120 bp, synthetic construct
  source 1..120: mol_type = other DNA; note = synthetic DNA probe; oligonucleotide; designed for Human Genome target region capture
gctttgtggccctcaaagtggtgaagagtgcggggcattacacggagacagctgtggataagatcaagctcctgaaatgtgtgaggcacctccctaccccactcccagctcccctggagc

SEQ ID NO: 36 - DNA, 120 bp, synthetic construct
  source 1..120: mol_type = other DNA; note = synthetic DNA probe; oligonucleotide; designed for Human Genome target region capture
agtccaggtctcttaccagggcagtggcgcccacgagggactccttggccagggcatggtgcagggcagagaacagccccatgctgttttgtctcagatagagcacctcgcccacgccgc

SEQ ID NO: 37 - DNA, 120 bp, synthetic construct
  source 1..120: mol_type = other DNA; note = synthetic DNA probe; oligonucleotide; designed for Human Genome target region capture
tcctagattttccacagaatgagcctcagatcaagaatcagtttaataagaagctatcaagaagacttgaaaatacaaaacagcaattgcagctgcctcttcatccttcatgggaagcaa